(19) United States
(12) Patent Application Publication
(10) Pub. No.: US 2009/0214561 A1
(43) Pub. Date: Aug. 27, 2009

(54) Treatment method

(76) Inventor: David Robert Close, Hertfordshire (GB)

Correspondence Address: FULBRIGHT & JAWORSKI, LLP, 666 FIFTH AVE, NEW YORK, NY, 10103-3198, US

(21) Appl. No.: 12/284,522

(22) Filed: Sep. 23, 2008

Related U.S. Application Data
(60) Provisional application No. 60/974,641, filed on Sep. 24, 2007.

(51) Int. Cl. A61K 39/395; C07K 16/28

(52) U.S. Cl. 424/144.1; 530/388.22

(57) ABSTRACT

The invention provides methods of treating autoimmune diseases by administration of a complete therapeutically effective amount of a CD20 specific antagonist as a single intravenous infusion. Retreatment may follow by the administration of a complete therapeutically effective amount of the CD20 specific antagonist by a single intravenous infusion, 4 to 6 months after the first administration. The CD20 specific antagonist preferably is a CD20 specific antibody, and the autoimmune disease preferably is rheumatoid arthritis.

### RELATED APPLICATION

This application claims priority from U.S. Provisional Application No. 60/974,641 filed Sep. 24, 2007, incorporated by reference in its entirety.

### FIELD OF THE INVENTION

The invention relates to the treatment of autoimmune diseases, including rheumatoid arthritis, by administration of a CD20 antibody, using a treatment regimen that includes a single intravenous infusion of the full effective dosage at the outset of treatment.

### BACKGROUND OF THE INVENTION

Lymphocytes are one of several populations of white blood cells; they specifically recognize and respond to foreign antigen. The three major classes of lymphocytes are B lymphocytes (B cells), T lymphocytes (T cells) and natural killer (NK) cells. B lymphocytes are the cells responsible for antibody production and provide humoral immunity. B cells mature within the bone marrow and leave the marrow expressing an antigen-binding antibody on their cell surface. When a naive B cell first encounters the antigen for which its membrane-bound antibody is specific, the cell begins to divide rapidly and its progeny differentiate into memory B cells and effector cells called “plasma cells”. Memory B cells have a longer life span and continue to express membrane-bound antibody with the same specificity as the original parent cell. Plasma cells do not produce membrane-bound antibody but instead produce a secreted form of the antibody. Secreted antibodies are the major effector molecules of humoral immunity.

The CD20 antigen (also called human B-lymphocyte-restricted differentiation antigen, Bp35) is a hydrophobic, transmembrane protein with a molecular weight of approximately 35 kD located on pre-B and mature B lymphocytes (Valentine et al. J. Biol. Chem. 264(19):11282-11287 (1989); and Einfeld et al. EMBO J. 7(3):711-717 (1988)). The antigen is also expressed on greater than 90% of B cell non-Hodgkin's lymphomas (NHL) (Anderson et al. Blood 63(6):1424-1433 (1984)), but is not found on hematopoietic stem cells, pro-B cells, normal plasma cells or other normal tissues (Tedder et al. J. Immunol. 135(2):973-979 (1985)). CD20 is thought to regulate an early step(s) in the activation process for cell cycle initiation and differentiation (Tedder et al., supra) and possibly functions as a calcium ion channel (Tedder et al. J. Cell. Biochem. 14D: 195 (1990)).

Given the expression of CD20 in B cell lymphomas, this antigen has been a useful therapeutic target to treat such lymphomas. There are more than 300,000 people in the United States with B-cell NHL and more than 56,000 new cases are diagnosed each year. CD20 is also a useful target antigen for treating autoimmune diseases.

The rituximab (RITUXAN®, Mabthera® in Europe) antibody, which is a genetically engineered chimeric murine/human monoclonal antibody directed against human CD20 antigen (commercially available from Genentech, Inc., South San Francisco, Calif., U.S.) is used for the treatment of patients with relapsed or refractory low-grade or follicular, CD20 positive, B cell non-Hodgkin's lymphoma. Rituximab is the antibody referred to as “C2B8” in U.S. Pat. No. 5,736,137 issued Apr. 7, 1998 (Anderson et al.) and in U.S. Pat. No. 5,776,456, both of which are incorporated by reference.

Rituximab has also been studied in a variety of non-malignant autoimmune disorders, in which B cells and autoantibodies appear to play a role in disease pathophysiology. Edwards et al., Biochem Soc. Trans. 30:824-828 (2002). Rituximab has been reported to potentially relieve signs and symptoms of, for example, rheumatoid arthritis (RA) (Leandro et al., Ann. Rheum. Dis. 61:883-888 (2002); Edwards et al., Arthritis Rheum., 46 (Suppl. 9): S46 (2002); Stahl et al., Ann. Rheum. Dis., 62 (Suppl. 1): OP004 (2003); Emery et al., Arthritis Rheum. 48(9): S439 (2003)), lupus (Eisenberg, Arthritis. Res. Ther. 5:157-159 (2003); Leandro et al Arthritis Rheum. 46: 2673-2677 (2002); Gorman et al., Lupus, 13: 312-316 (2004)), immune thrombocytopenic purpura (D'Arena et al., Leuk. Lymphoma 44:561-562 (2003); Stasi et al., Blood, 98: 952-957 (2001); Saleh et al., Semin. Oncol., 27 (Supp 12):99-103 (2000); Zaja et al., Haematolgica, 87: 189-195 (2002); Ratanatharathom et al., Ann. Int. Med., 133: 275-279 (2000)), pure red cell aplasia (Auner et al, Br. J. Haematol., 116: 725-728 (2002)); autoimmune anemia (Zaja et al., Haematologica 87:189-195 (2002) (erratum appears in Haematologica 87:336 (2002)), cold agglutinin disease (Layios et al., Leukemia, 15: 187-8 (2001); Berentsen et al., Blood, 103: 2925-2928 (2004); Berentsen et al., Br. J. Haematol., 115: 79-83 (2001); Bauduer, Br. J. Haematol., 112: 1083-1090 (2001); Zaja et al., Br. J. Haematol., 115: 232-233 (2001)), type B syndrome of severe insulin resistance (Coll et al., N. Engl. J. Med., 350: 310-311 (2004), mixed cryoglobulinemia (DeVita et al., Arthritis Rheum. 46 Suppl. 9:S206/S469 (2002)), myasthenia gravis (Zaja et al., Neurology, 55: 1062-63 (2000); Wylam et al., J. Pediatr., 143: 674-677 (2003)), Wegener's granulomatosis (Specks et al., Arthritis & Rheumatism 44: 2836-2840 (2001)), refractory pemphigus vulgaris (Dupuy et al., Arch Dermatol., 140:91-96 (2004)), demmatomyositis (Levine, Arthritis Rheum., 46 (Suppl. 9):S1299 (2002)), Sjogren's syndrome (Somer et al., Arthritis & Rheumatism, 49: 394-398 (2003)), active type-II mixed cryoglobulinemia (Zaja et al., Blood, 101: 3827-3834 (2003)), pemphigus vulgaris (Dupay et al., Arch. Dermatol., 140: 91-95 (2004)), autoimmune neuropathy (Pestronk et al., J. Neurol. Neurosurg. Psychiatry 74:485-489 (2003)), paraneoplastic opsoclonus-myoclonus syndrome (Pranzatelli et al. Neurology 60(Suppl. 1) PO5.128:A395 (2003)), and relapsing-remitting multiple sclerosis (RRMS). Cross et al. (abstract) “Preliminary Results from a Phase II Trial of Rituximab in MS” Eighth Annual Meeting of the Americas Committees for Research and Treatment in Multiple Sclerosis, 20-21 (2003).

A Phase II study (WA 16291) has been conducted in patients with rheumatoid arthritis (RA), providing 48-week follow-up data on safety and efficacy of Rituximab. Emery et al. Arthritis Rheum 48(9):S439 (2003); Szczepanski et al. Arthritis Rheum 48(9):S121 (2003). A total of 161 patients were randomized to four treatment arms: methotrexate, rituximab alone, rituximab plus methotrexate, and rituximab plus cyclophosphamide (CTX). The treatment regimen of rituximab was one gram administered intravenously on days 1 and 15. Infusions of rituximab in most patients with RA were well tolerated by most patients, with 36% of patients experiencing at least one adverse event during their first infusion (compared with 30% of patients receiving placebo). Overall, the majority of adverse events was considered to be mild to moderate in severity and was well balanced across all treatment groups. There were a total of 19 serious adverse events across the four arms over the 48 weeks, which were slightly more frequent in the rituximab/CTX group. The incidence of infections was well balanced across all groups. The mean rate of serious infection in this RA patient population was 4.66 per 100 patient-years, which is lower than the rate of infections requiring hospital admission in RA patients (9.57 per 100 patient-years) reported in a community-based epidemiologic study. Doran et al., Arthritis Rheum. 46:2287-2293 (2002).

The reported safety profile of rituximab in a small number of patients with neurologic disorders, including autoimmune neuropathy (Pestronk et al., supra), opsoclonus-myoclonus syndrome (Pranzatelli et al., supra), and RRMS (Cross et al., supra), was similar to that reported in oncology or RA. In an ongoing investigator-sponsored trial (IST) of rituximab in combination with interferon-beta (IFN-3) or glatiramer acetate in patients with RRMS (Cross et al., supra), 1 of 10 treated patients was admitted to the hospital for overnight observation after experiencing moderate fever and rigors following the first infusion of rituximab, while the other 9 patients completed the four-infusion regimen without any reported adverse events.

Patents and patent publications concerning CD20 antibodies, CD20-binding molecules, and self-antigen vaccines include U.S. Pat. Nos. 5,776,456, 5,736,137, 5,843,439, 6,399,061, and 6,682,734, as well as US 2002/0197255, US 2003/0021781, US 2003/0082172, US 2003/0095963, US 2003/0147885, US 2005/0186205, and WO 1994/11026 (Anderson et al.); U.S. Pat. No. 6,455,043, US 2003/0026804, US 2003/0206903, and WO 2000/09160 (Grillo-Lopez, A.); WO 2000/27428 (Grillo-Lopez and White); US 2004/0213784 and WO 2000/27433 (Grillo-Lopez and Leonard); WO 2000/44788 (Braslawsky et al.); WO 2001/10462 (Rastetter, W.); WO 2001/10461 (Rastetter and White); WO 2001/10460 (White and Grillo-Lopez); US 2001/0018041, US 2003/0180292, US 2002/0028178, WO 2001/34194, and WO 2002/22212 (Hanna and Hariharan); US 2002/0006404 and WO 2002/04021 (Hanna and Hariharan); US 2002/0012665, US 2005/0180975, WO 2001/74388, and U.S. Pat. No. 6,896,885B5 (Hanna, N.); US 2002/0058029 (Hanna, N.); US 2003/0103971 (Hariharan and Hanna); US 2005/0123540 (Hanna et al.); US 2002/0009444 and WO 2001/80884 (Grillo-Lopez, A.); WO 2001/97858; US 2005/0112060, US 2002/0039557, and U.S. Pat. No. 6,846,476 (White, C.); US 2002/0128448 and WO 2002/34790 (Reff, M.); WO 2002/060955 (Braslawsky et al.); WO 2002/096948 (Braslawsky et al.); WO 2002/079255 (Reff and Davies); U.S. Pat. Nos. 6,171,586 and 6,991,790, and WO 1998/56418 (Lam et al.); US 2004/0191256 and WO 1998/58964 (Raju, S.); WO 1999/22764 (Raju, S.); WO 1999/51642, U.S. Pat. No. 6,194,551, U.S. Pat. No. 6,242,195, U.S. Pat. No. 6,528,624 and U.S. Pat. No. 6,538,124 (Idusogie et al.); U.S. Pat. No. 7,122,637, US 2005/0118174, US 2005/0233382, US 2006/0194291, US 2006/0194290, US 2006/0194957, and WO 2000/42072 (Presta, L.); WO 2000/67796 (Curd et al.); WO 2001/03734 (Grillo-Lopez et al.); US 2002/0004587, US 2006/0025576, and WO 2001/77342 (Miller and Presta); US 2002/0197256 and WO 2002/078766 (Grewal, I.); US 2003/0157108 and WO 2003/035835 (Presta, L.); U.S. Pat. Nos. 5,648,267, 5,733,779, 6,017,733, and 6,159,730, and WO 1994/11523 (Reff et al. on expression technology); U.S. Pat. Nos. 6,565,827, 6,090,365, 6,287,537, 6,015,542, 5,843,398, and 5,595,721 (Kaminski et al.); U.S. Pat. Nos. 5,500,362, 5,677,180, 5,721,108, 6,120,767, 6,652,852, and 6,893,625 as well as WO 1988/04936 (Robinson et al.); U.S. Pat. No. 6,410,391 (Zelsacher); U.S. Pat. No. 6,224,866 and WO00/20864 (Barbera-Guillem, E.); WO 2001/13945 (Barbera-Guillem, E.); WO 2000/67795 (Goldenberg); U.S. Pat. No. 7,074,403 (Goldenberg and Hansen); U.S. Pat. No. 7,151,164 (Hansen et al.); US 2003/0133930; WO 2000/74718 and US 2005/0191300A1 (Goldenberg and Hansen); US 2003/0219433 and WO 2003/68821 (Hansen et al.); WO 2004/058298 (Goldenberg and Hansen); WO 2000/76542 (Golay et al.); WO 2001/72333 (Wolin and Rosenblatt); U.S. Pat. No. 6,368,596 (Ghetie et al.); U.S. Pat. No. 6,306,393 and US 2002/0041847 (Goldenberg, D.); US 2003/0026801 (Weiner and Hartmann); WO 2002/102312 (Engleman, E.); US 2003/0068664 (Albitar et al.); WO 2003/002607 (Leung, S.); WO 2003/049694, US 2002/0009427, and US 2003/0185796 (Wolin et al.); WO 2003/061694 (Sing and Siegall); US 2003/0219818 (Bohen et al.); US 2003/0219433 and WO 2003/068821 (Hansen et al.); US 2003/0219818 (Bohen et al.); US 2002/0136719 (Shenoy et al.); WO 2004/032828 and US 2005/0180972 (Wahl et al.); and WO 2002/56910 (Hayden-Ledbetter). See also U.S. Pat. No. 5,849,898 and EP 330,191 (Seed et al.); EP332,865A2 (Meyer and Weiss); U.S. Pat. No. 4,861,579 (Meyer et al.); US 2001/0056066 (Bugelski et al.); WO 1995/03770 (Bhat et al.); US 2003/0219433 A1 (Hansen et al.); WO 2004/035607 and US 2004/167319 (Teeling et al.); WO 2005/103081 (Teeling et al.); US 2006/0034835, US 2006/0024300, and WO 2004/056312 (Lowman et al.); US 2004/0093621 (Shiara et al.); WO 2004/103404 (Watkins et al.); WO 2005/000901 (Tedder et al.); US 2005/0025764 (Watkins et al.); US 2006/0251652 (Watkins et al.); WO 2005/016969 (Carr et al.); US 2005/0069545 (Carr et al.); WO 2005/014618 (Chang et al.); US 2005/0079174 (Barbera-Guillem and Nelson); US 2005/0106108 (Leung and Hansen); US 2005/0123546 (Umana et al.); US 2004/0072290 (Umana et al.); US 2003/0175884 (Umana et al.); and WO 2005/044859 (Umana et al.); WO 2005/070963 (Allan et al.); US 2005/0186216 (Ledbetter and Hayden-Ledbetter); US 2005/0202534 (Hayden-Ledbetter and Ledbetter); US 2005/136049 (Ledbetter et al.); US 2003/118592 (Ledbetter et al.); US 2003/133939 (Ledbetter and Hayden-Ledbetter); US 2005/0202012 (Ledbetter and Hayden-Ledbetter); US 2005/0175614 (Ledbetter and Hayden-Ledbetter); US 2005/0180970 (Ledbetter and Hayden-Ledbetter); US 2005/0202028 (Hayden-Ledbetter and Ledbetter); US 2005/0202023 (Hayden-Ledbetter and Ledbetter); WO 2005/017148 (Ledbetter et al.); WO 2005/037989 (Ledbetter et al.); U.S. Pat. No. 6,183,744 (Goldenberg); U.S. Pat. No. 6,897,044 (Braslawski et al.); WO 2006/005477 (Krause et al.); US 2006/0029543 (Krause et al.); US 2006/0018900 (McCormick et al.); US 2006/0051349 (Goldenberg and Hansen); WO 2006/042240 (Iyer and Dunussi-Joannopoulos); US 2006/0121032 (Dahiyat et al.); WO 2006/064121 (Teillaud et al.); US 2006/0153838 (Watkins), CN 1718587 (Chen et al.); WO 2006/084264 (Adams et al.); US 2006/0188495 (Barron et al.); US 2004/0202658 and WO 2004/091657 (Benynes, K.); US 2005/0095243, US 2005/0163775, WO 2005/00351, and WO 2006/068867 (Chan, A.); US 2006/0135430 and WO 2005/005462 (Chan et al.); US 2005/0032130 and WO 2005/017529 (Beresini et al.); US 2005/0053602 and WO 2005/023302 (Brunetta, P.); US 2006/0179501 and WO 2004/060052 (Chan et al.); WO 2004/060053 (Chan et al.); US 2005/0186206 and WO 2005/060999 (Brunetta, P.); US 2005/0191297 and WO 2005/061542 (Brunetta, P.); US 2006/0002930 and WO 2005/115453 (Brunetta et al.); US 2006/0099662 and WO 2005/108989 (Chuntharapai et al.); CN 1420129A (Zhongxin Guojian Pharmaceutical); US 2005/0276803 and WO 2005/113003 (Chan et al.); US 2005/0271658 and WO 2005/117972 (Brunetta et al.); US 2005/0255527 and WO 2005/11428 (Yang, J.); US 2006/0024295 and WO 2005/120437 (Brunetta, P.); US 2006/0051345 and WO 2005/117978 (Frohna, P.); US 2006/0062787 and WO 2006/012508 (Hitraya, E.); US 2006/0067930 and WO 2006/31370 (Lowman et al.); WO 2006/29224 (Ashkenazi, A.); US 2006/0110387 and WO 2006/41680 (Brunetta, P.); US 2006/0134111 and WO 2006/066086 (Agarwal, S.); WO 2006/069403 (Ernst and Yansura); US 2006/0188495 and WO 2006/076651 (Dummer, W.); WO 2006/084264 (Lowman, H.); WO 2006/093923 (Quan and Sewell); WO 2006/106959 (Numazaki et al.); WO 2006/126069 (Morawala); WO 2006/130458 (Gazit-Bornstein et al.); US 2006/0275284 (Hanna, G.); US 2007/0014785 (Golay et al.); US 2007/0014720 (Gazit-Bornstein et al.); and US 2007/0020259 (Hansen et al.); US 2007/0020265 (Goldenberg and Hansen); US 2007/0014797 (Hitraya); US 2006/0246004 and WO 06/084264.

Scientific publications concerning treatment with rituximab include: Perotta and Abuel, “Response of chronic relapsing ITP of 10 years duration to rituximab” Abstract # 3360 Blood 10(1)(part 1-2): p. 88B (1998); Perotta et al., “Rituxan in the treatment of chronic idiopathic thrombocytopaenic purpura (ITP)”, Blood, 94: 49 (abstract) (1999); Matthews, R., “Medical Heretics” New Scientist (7 Apr., 2001); Leandro et al., “Clinical outcome in 22 patients with rheumatoid arthritis treated with B lymphocyte depletion” Ann Rheum Dis, supra; Leandro et al., “Lymphocyte depletion in rheumatoid arthritis: early evidence for safety, efficacy and dose response” Arthritis and Rheumatism 44(9): S370 (2001); Leandro et al., “An open study of B lymphocyte depletion in systemic lupus erythematosus”, Arthritis and Rheumatism, 46:2673-2677 (2002), wherein during a 2-week period, each patient received two 500-mg infusions of rituximab, two 750-mg infusions of cyclophosphamide, and high-dose oral corticosteroids, and wherein two of the patients treated relapsed at 7 and 8 months, respectively, and have been retreated, although with different protocols; “Successful long-term treatment of systemic lupus erythematosus with rituximab maintenance therapy” Weide et al., Lupus, 12: 779-782 (2003), wherein a patient was treated with rituximab (375 mg/m²×4, repeated at weekly intervals) and further rituximab applications were delivered every 5-6 months and then maintenance therapy was received with rituximab 375 mg/m² every three months, and a second patient with refractory SLE was treated successfully with rituximab and is receiving maintenance therapy every three months, with both patients responding well to rituximab therapy; Edwards and Cambridge, “Sustained improvement in rheumatoid arthritis following a protocol designed to deplete B lymphocytes” Rheumatology 40:205-211 (2001); Cambridge et al., “B lymphocyte depletion in patients with rheumatoid arthritis: serial studies of immunological parameters” Arthritis Rheum., 46 (Suppl. 9): S1350 (2002); Cambridge et al., “Serologic changes following B lymphocyte depletion therapy for rheumatoid arthritis” Arthritis Rheum., 48: 2146-2154 (2003); Edwards et al., “B-lymphocyte depletion therapy in rheumatoid arthritis and other autoimmune disorders” Biochem Soc. Trans., supra; Edwards et al., “Efficacy and safety of rituximab, a B-cell targeted chimeric monoclonal antibody: A randomized, placebo controlled trial in patients with rheumatoid arthritis. Arthritis and Rheumatism 46(9): S197 (2002); Edwards et al., “Efficacy of B-cell-targeted therapy with rituximab in patients with rheumatoid arthritis” N Engl. J. Med. 350:2572-2582 (2004); Pavelka et al., Ann. Rheum. Dis. 63: (S1):289-290 (2004); Emery et al., Arthritis Rheum. 50 (S9):S659 (2004); Levine and Pestronk, “IgM antibody-related polyneuropathies: B-cell depletion chemotherapy using rituximab” Neurology 52: 1701-1704 (1999); Uchida et al., “The innate mononuclear phagocyte network depletes B lymphocytes through Fc receptor-dependent mechanisms during anti-CD20 antibody immunotherapy” J. Exp. Med. 199: 1659-1669 (2004); Gong et al., “Importance of cellular microenvironment and circulatory dynamics in B cell immunotherapy” J. Immunol. 174: 817-826 (2005); Hamaguchi et al., “The peritoneal cavity provides a protective niche for B1 and conventional B lymphocytes during anti-CD20 immunotherapy in mice” J. Immunol. 174: 4389-4399 (2005); Cragg et al. “The biology of CD20 and its potential as a target for mAb therapy” Curr. Dir. Autoimmun. 8:140-174 (2005); Eisenberg, “Mechanisms of autoimmunity” Immunol. Res. 27: 203-218 (2003); DeVita et al., “Efficacy of selective B cell blockade in the treatment of rheumatoid arthritis” Arthritis & Rheum 46:2029-2033 (2002); Higashida et al. “Treatment of DMARD-refractory rheumatoid arthritis with rituximab.” Presented at the Annual Scientific Meeting of the American College of Rheumatology; Oct. 24-29; (Abstract #LB11), New Orleans, La. 2002; Tuscano, J. “Successful treatment of infliximab-refractory rheumatoid arthritis with rituximab” Presented at the Annual Scientific Meeting of the American College of Rheumatology; Oct. 24-29; New Orleans, La. 2002 and published Tuscano, Arthritis Rheum. 46: 3420 (2002); “Pathogenic roles of B cells in human autoimmunity; insights from the clinic” Martin and Chan, Immunity 20:517-527 (2004); Silverman and Weisman, “Rituximab therapy and autoimmune disorders, prospects for anti-B cell therapy”, Arthritis and Rheumatism, 48: 1484-1492 (2003); Kazkaz and Isenberg, “Anti B cell therapy (rituximab) in the treatment of autoimmune diseases”, Current opinion in pharmacology, 4: 398-402 (2004); Virgolini and Vanda, “Rituximab in autoimmune diseases”, Biomedicine & pharmacotherapy, 58: 299-309 (2004); Klemmer et al., “Treatment of antibody mediated autoimmune disorders with a AntiCD20 monoclonal antibody Rituximab”, Arthritis And Rheumatism, 48: (9) 9,S (SEP), page: S624-S624 (2003); Kneitz et al., “Effective B cell depletion with rituximab in the treatment of autoimmune diseases”, Immunobiology, 206: 519-527 (2002); Arzoo et al., “Treatment of refractory antibody mediated autoimmune disorders with an anti-CD20 monoclonal antibody (rituximab)” Annals of the Rheumatic Diseases, 61 (10), p 922-924 (2002) Comment in Ann Rheum Dis. 61: 863-866 (2002); “Future strategies in immunotherapy” by Lake and Dionne, in Burger's Medicinal Chemistry and Drug Discovery (2003 by John Wiley & Sons, Inc.) Article Online Posting Date Jan. 15, 2003 (Chapter 2“Antibody-Directed Immunotherapy”); Liang and Tedder, Wiley Encyclopedia of Molecular Medicine, Section: CD20 as an Immunotherapy Target, article online posting date: 15 Jan., 2002 entitled “CD20”; Appendix 4A entitled “Monoclonal Antibodies to Human Cell Surface Antigens” by Stockinger et al., eds: Coligan et al., in Current Protocols in Immunology (2003 John Wiley & Sons, Inc) Online Posting Date: May, 2003; Print Publication Date: February, 2003; Penichet and Morrison, “CD Antibodies/molecules: Definition; Antibody Engineering” in Wiley Encyclopedia of Molecular Medicine Section: Chimeric, Humanized and Human Antibodies; posted online 15 Jan., 2002.

Further, see Looney “B cells as a therapeutic target in autoimmune diseases other than rheumatoid arthritis” Rheumatology, 44 Suppl 2: ii13-ii17 (2005); Chambers and Isenberg, “Anti-B cell therapy (rituximab) in the treatment of autoimmune diseases” Lupus 14(3): 210-214 (2005); Looney et al., “B-cell depletion as a novel treatment for systemic lupus erythematosus: a phase I/II dose-escalating trial of rituximab” Arthritis Rheum. 50: 2580-2589 (2004); Looney, “Treating human autoimmune disease by depleting B cells” Ann Rheum. Dis. 61: 863-866 (2002); Edelbauer et al., “Rituximab in childhood systemic lupus erythematosus refractory to conventional immunosuppression Case report” Pediatr. Nephrol. 20(6): 811-813 (2005); D'Cruz and Hughes, “The treatment of lupus nephritis” BMJ 330(7488): 377-378 (2005); Looney, “B cell-targeted therapy in diseases other than rheumatoid arthritis” J. Rheumatol. Suppl. 73: 25-28; discussion 29-30 (2005); Sfikakis et al., “Remission of proliferative lupus nephritis following B cell depletion therapy is preceded by down-regulation of the T cell costimulatory molecule CD40 ligand: an open-label trial” Arthritis Rheum. 52(2): 501-513 (2005); Rastetter et al., “Rituximab: expanding role in therapy for lymphomas and autoimmune diseases” Annu. Rev. Med. 55: 477-503 (2004); Silverman, “Anti-CD20 therapy in systemic lupus erythematosus: a step closer to the clinic” Arthritis Rheum. 52(2): 371-7 (2005), Erratum in: Arthritis Rheum. 52(4): 1342 (2005); Ahn et al., “Long-term remission from life-threatening hypercoagulable state associated with lupus anticoagulant (LA) following rituximab therapy” Am. J. Hematol. 78(2): 127-129 (2005); Tahir et al., “Humanized anti-CD20 monoclonal antibody in the treatment of severe resistant systemic lupus erythematosus in a patient with antibodies against rituximab” Rheumatology, 44(4): 561-562 (2005), Epub 2005 Jan. 11; Looney et al., “Treatment of SLE with anti-CD20 monoclonal antibody” Curr. Dir. Autoimmun. 8: 193-205 (2005); Cragg et al., “The biology of CD20 and its potential as a target for mAb therapy” Curr. Dir. Autoimmun. 8: 140-174 (2005); Gottenberg et al., “Tolerance and short term efficacy of rituximab in 43 patients with systemic autoimmune diseases” Ann. Rheum. Dis. 64(6): 913-920 (2005) Epub 2004 Nov. 18; Tokunaga et al., “Down-regulation of CD40 and CD80 on B cells in patients with life-threatening systemic lupus erythematosus after successful treatment with rituximab” Rheumatology 44(2): 176-182 (2005), Epub 2004 Oct. 19. See also Leandro et al., “B cell repopulation occurs mainly from naïve B cells in patient with rheumatoid arthritis and systemic lupus erythematosus” Arthritis Rheum., 48 (Suppl 9): S1160 (2003).

Specks et al. “Response of Wegener's granulomatosis to anti-CD20 chimeric monoclonal antibody therapy” Arthritis & Rheumatism 44(12):2836-2840 (2001) discloses successful use of four infusions of 375 mg/m² of rituximab and high-dose glucocorticoids to treat Wegener's granulomatosis. The therapy was repeated after 11 months when the cANCA recurred, but therapy was without glucocorticoids. At 8 months after the second course of rituximab, the patients' disease remained in complete remission. Further, in another study, rituximab was found to be a well-tolerated, effective remission induction agent for severe ANCA-associated vasculitis, when used in a dose of 375 mg/m²×4 along with oral prednisone 1 mg/kg/day, which was reduced by week 4 to 40 mg/day, and to complete discontinuation over the following 16 weeks. Four patients were re-treated with rituximab alone for recurring/rising ANCA titers. Other than glucocorticoids, no additional immunosuppressive agents seem to be necessary for remission induction and maintenance of sustained remission (6 months or longer). Keogh et al., Kidney Blood Press. Res. 26:293 (2003) reported that eleven patients with refractory ANCA-associated vasculitis were treated with four weekly doses of 375 mg/m of rituximab and high-dose glucocortoicoids, resulting in remission.

Patients with refractory ANCA-associated vasculitis were administered rituximab along with immunosuppressive medicaments such as intravenous cyclophosphamide, mycophenolate mofetil, azathioprine, or leflunomide, with apparent efficacy. Eriksson, “Short-term outcome and safety in 5 patients with ANCA-positive vasculitis treated with rituximab”, Kidney and Blood Pressure Research, 26: 294 (2003) (five patients with ANCA-associated vasculitis treated with rituximab 375 mg/m² once a week for 4 weeks responded to the treatment); Jayne et al., “B-cell depletion with rituximab for refractory vasculitis” Kidney and Blood Pressure Research, 26: 294-295 (2003) (six patients with refractory vasculitis receiving four weekly infusions of rituximab at 375 mg/m² with cyclophosphamide along with background immunosuppression and prednisolone experienced major falls in vasculitic activity). A further report of using rituximab along with intravenous cyclophosphamide at 375 mg/m² per dose in 4 doses for administering to patients with refractory systemic vasculitis is provided in Smith and Jayne, A prospective, open label trial of B-cell depletion with rituximab in refractory systemic vasculitis, poster 998 (11^{th} International Vasculitis and ANCA workshop), American Society of Nephrology, J. Am. Soc. Nephrol., 14: p. 755A (2003). See also Eriksson, J. Internal Med., 257: 540-548 (2005) regarding nine patients with ANCA-positive vasculitis who were successfully treated with two or four weekly doses of 500 mg of rituximab, as well as Keogh et al., Arthritis and Rheumatism, 52: 262-268 (2005), who reported that in 11 patients with refractory ANCA-associated vasculitis, treatment or re-treatment with four weekly doses of 375 mg/m² of rituximab induced remission by B lymphocyte depletion, the study being conducted between January 2000 and September 2002.

As to the activity of a humanized anti-CD20 antibody, see, for example, Vugmeyster et al., “Depletion of B cells by a humanized anti-CD20 antibody PRO70769 in Macaca fascicularis” J. Immunother. 28: 212-219 (2005). For discussion of a human monoclonal antibody, see Baker et al., “Generation and characterization of LymphoStat-B, a human monoclonal antibody that antagonizes the bioactivities of B lymphocyte stimulator” Arthritis Rheum. 48: 3253-3265 (2003). Further, the MINT trial with rituximab was successful in treating aggressive non-Hodgkin's lymphoma in younger patients Lancet Oncology (Apr. 5, 2006).

Trubion Pharmaceuticals Inc. has recently evaluated the safety and efficacy of an anti-CD20 antibody fragment-type molecule designated TRU-015 in a Phase IIb randomized, double-blind, placebo-controlled, multicenter clinical trial conducted with 276 patients with rheumatoid arthritis who were also receiving methotrexate as background therapy. The patients were randomized evenly into five groups that received either placebo, 200 mg, 400 mg, 800 mg or 1600 mg of TRU-015, administered as a single intravenous infusion. TRU-015 was reported to be generally well tolerated.

In treating a disease, it is beneficial to be able to administer the drug in a way that is both efficacious, safe and well tolerated by the patients, with little or no side-effects, and can be advantageously implemented in everyday clinical practice, with as little inconvenience for the patient as possible. As will be apparent from the detailed description below, the present invention satisfies this need by providing a new protocol for the administration of CD20 antibodies for the treatment of autoimmune diseases.

### SUMMARY OF THE INVENTION

In one aspect, the invention concerns a method of depleting B cells in a human patient having an autoimmune disease comprising administering to the patient a complete therapeutically effective amount of a CD20 antagonist as a single intravenous (i.v.) infusion.

In one embodiment, the CD20 antagonist is a CD20 monoclonal antibody.

In a further embodiment, administration of the complete therapeutically effective amount of a CD20 antagonist as a single intravenous (i.v.) infusion is followed by administration of a second complete therapeutically effective amount by i.v. infusion, 4-6 months after the first administration. The second administration usually takes place when the patient treated responds to the first treatment but shows signs of relapse.

In another embodiment, the patient treated showed inadequate response to previously administered methotrexate, TNF antagonists, and/or other anti-CD20 antagonists, including CD20 antibodies other than the antibody administered in accordance with the present invention.

In yet another embodiment, the CD20 monoclonal antibody is chimeric, humanized or human.

In a further embodiment, the autoimmune disease is selected from the group consisting of rheumatoid arthritis, systemic lupus erythematosus (SLE), lupus nephritis, ulcerative colitis, Crohn's disease, Sjorgen's syndrome, neuormyelitis optica (NMO), ANCA associated-vasculitis, Wegener's disease, inflammatory bowel disease, idiopathic thrombocytopenic purpura (ITP), thrombotic thrombocytopenic purpura (TTP), autoimmune thrombocytopenia, multiple sclerosis, psoriasis, IgA nephropathy, IgM polyneuropathies, myasthenia gravis, diabetes mellitus, Reynaud's syndrome, and glomerulonephritis.

In a still further embodiment, the autoimmune disease is active rheumatoid arthritis, such as, for example, moderate to severe rheumatoid arthritis.

The therapeutically effective amount is typically between 10 mg and 2000 mg, preferably between about 400 mg and about 1500 mg. In separate embodiments, the therapeutically effective amount is 400 mg, 1000 mg, or 1500 mg.

In a preferred embodiment, the autoimmune disease is rheumatoid arthritis and the CD20 antibody is a humanized 2H7 antibody.

In various embodiments, the humanized CD20 antibody (1) is selected from the group consisting of 2H7 Variants A, B and I, comprising a full length L chain of SEQ ID NO: 6 and a full length heavy chain of SEQ ID NO: 7, 8 and 15, respectively, or a fragment thereof; or (2) is 2H7 Variant A comprising a full length L chain of SEQ ID NO: 6 and a full length H chain of SEQ ID NO: 7, or a fragment thereof; or (3) an antibody that binds to essentially the same epitope as 2H7 Variant A comprising a full length L chain of SEQ ID NO: 6 and a full length H chain of SEQ ID NO: 7, or a fragment thereof; or (4) is selected from the group consisting of 2H7 Variants C, D, F, G, and H comprising a full length L chain of SEQ ID NO: 9 and a full length H chain of SEQ ID NO: 10, 11, 12, 13, and 14 respectively, or a fragment thereof; of (5) is 2H7 Variant B comprising a full length L chain of SEQ ID NO: 6 and a full length H chain of SEQ ID NO: 8, or a fragment thereof; or (6) is 2H7 Variant C comprising a full length L chain of SEQ ID NO: 9 and a full length H chain of SEQ ID NO: 10, or a fragment thereof.

In one particular embodiment, the antibody is the chimeric antibody Rituximab.

In another embodiment, the CD20 antibody is the human antibody HUMAX-CD20.

In a further embodiment, the CD20 antibody is administered in conjunction with therapy using a drug selected from the group consisting of nonsteroidal anti-inflammatory drugs (NSAIDs), analgesics, glucocorticosteroids, cyclophosphamide, adalimumab, leflunomide, infliximab, etanercept, ofatumumab, tocilizumab, AME-133, Immu-106, and COX-2 inhibitors.

In a still further embodiment, the method comprises administering to the patient a second therapeutic agent, which can, for example, be an immunosuppressive agent.

In a preferred embodiment the patient receives concomitant methotrexate treatment.

In another embodiment, the patient had been administered one to six DMARDS prior to administration of the CD20 antibody.

In yet another embodiment, the patient did not receive any steroid treatment prior to the administration of the CD20 antibody.

In another aspect, the invention concerns a method for the treatment of active rheumatoid arthritis (RA) in a human patient comprising administering to the patient 400 mg to 1500 mg of a CD20 antibody as a single intravenous (i.v.) infusion.

Just as before, the antibody can be a chimeric, humanized or human monoclonal antibody, and may, for example, be any of the CD20 antibodies listed above.

In a particular embodiment, administration of the CD20 antibody is not followed by a second administration of a CD20 antibody for at least 1 month, or at least 2 months, or at least 3 months, or at least 4 months.

In a further aspect, the invention concerns a method for the treatment of active rheumatoid arthritis (RA) in a human patient comprising administering to the patient 400 mg of 2H7 Variant A comprising a full length L chain of SEQ ID NO: 6 and a full length H chain of SEQ ID NO: 7, or a fragment thereof.

In a still further aspect, the invention concerns a method for the treatment of active rheumatoid arthritis (RA) in a human patient comprising administering to the patient 1000 mg of CD20 antibody 2H7 Variant A comprising a full length L chain of SEQ ID NO: 6 and a full length H chain of SEQ ID NO: 7, or a fragment thereof.

In a particular embodiment, the first administration is not followed by a second administration of a CD20 antibody for at least 1 month, or at least 2 months, or at least 3 months, or at least 4 months.

In all methods herein, the initial infusion rate may be within the normal standard range of infusion rates, such as, for example, between 50 mg/hr and 100 mg/hr. The infusion rate does not need to be the same during the entire time of administration, rather can be escalated, e.g. as described in Example 1.

In a different aspect, the invention concerns an article of manufacture comprising: (a) a container comprising a CD20 antagonist; and (b) a package insert with instructions for treating an autoimmune disease in a human subject, wherein the instructions indicate that the subject is administered the complete therapeutically effective amount of the CD20 antagonist as single intravenous infusion.

In a further aspect, the invention concerns a pharmaceutical formulation comprising a complete effective amount of a CD20 antibody in a form suitable for one-time intravenous administration.

### DETAILED DESCRIPTION OF THE PREFERRED EMBODIMENTS

### I. Definitions

A “B cell” is a lymphocyte that matures within the bone marrow, and includes a naive B cell, memory B cell, or effector B cell (plasma cells), in all stages of development. The B cell herein is a normal or non-malignant B cell.

As used herein, “B cell depletion” refers to a reduction in B cell levels in an animal or human after drug or antibody treatment, as compared to the level before treatment. B cell levels are measurable using well known assays such as by getting a complete blood count, by FACS analysis staining for known B cell markers, and other methods well known in the art.

In one embodiment, the depletion of CD20 expressing B cells is at least 25%. In a patient receiving a B cell depleting drug, B cells are generally depleted for the duration of time when the drug is circulating in the patient's body and the time for recovery of B cells.

A “B-cell surface marker” or “B-cell surface antigen” or “B cell antigen” herein is an antigen expressed on the surface of a B cell that can be targeted with an antagonist that binds thereto. Exemplary B-cell surface markers include the CD10, CD19, CD20, CD21, CD22, CD23, CD24, CD37, CD40, CD53, CD72, CD73, CD74, CDw75, CDw76, CD77, CDw78, CD79a, CD79b, CD80, CD81, CD82, CD83, CDw84, CD85 and CD86 leukocyte surface markers (for descriptions, see The Leukocyte Antigen Facts Book, 2^{nd} Edition. 1997, ed. Barclay et al. Academic Press, Harcourt Brace & Co., New York). Other B-cell surface markers include RP105, FcRH2, B-cell CR2, CCR6, P2×5, HLA-DOB, CXCR5, FCER2, BR3, Btig, NAG14, SLGC16270, FcRH1, IRTA2, ATWD578, FcRH3, IRTAl, FcRH6, BCMA, and 239287. The B-cell surface marker of particular interest is preferentially expressed on B cells compared to other non-B-cell tissues of a mammal and may be expressed on both precursor B cells and mature B cells. The preferred B-cell surface markers herein are CD20 and CD22.

The “CD20” antigen, or “CD20,” is an about 35-kDa, non-glycosylated phosphoprotein found on the surface of greater than 90% of B cells from peripheral blood or lymphoid organs. CD20 is present on both normal B cells as well as malignant B cells, but is not expressed on stem cells. Other names for CD20 in the literature include “B-lymphocyte-restricted antigen” and “Bp35”. The CD20 antigen is described in Clark et al., Proc. Natl. Acad. Sci. (USA) 82:1766 (1985), for example.

A “CD20 antagonist” is a molecule that, upon binding to CD20 on B cells, destroys or depletes B cells in a mammal and/or interferes with one or more B-cell functions, e.g. by reducing or preventing a humoral response elicited by the B cell. The CD20 antagonist preferably is able to deplete B cells (i.e. reduce circulating B-cell levels) in a mammal, such as a human subject, treated therewith. Such depletion may be achieved via various mechanisms such as ADCC and/or CDC, inhibition of B-cell proliferation and/or induction of B-cell death (e.g. via apoptosis). Antagonists included within the scope of the present invention include antibodies, other CD20 targeted (binding) proteins, synthetic or native-sequence peptides, immunoadhesins, and small-molecule antagonists that bind to CD20, optionally conjugated with or fused to another molecule. The preferred antagonist is, consists of, consists essentially of or comprises an antibody.

An “antibody antagonist” herein is an antibody that, upon binding to a B-cell surface marker on B cells, destroys or depletes B cells in a mammal and/or interferes with one or more B-cell functions, e.g., by reducing or preventing a humoral response elicited by the B cell. The antibody antagonist preferably is able to deplete B cells (i.e., reduce circulating B-cell levels) in a mammal treated therewith. Such depletion may be achieved via various mechanisms such as ADCC and/or CDC, inhibition of B-cell proliferation and/or induction of B-cell death (e.g., via apoptosis). Thus, a “CD20 antibody antagonist” or an “antagonist CD20 antibody” is an antibody that, upon binding to CD20 on B cells, destroys or depletes B cells in a mammal, such as a human subject, and/or interferes with one or more B-cell functions, e.g., by reducing or preventing a humoral response elicited by the B cell. CD20 antagonist antibodies include rituximab (Genentech), TRU-015 (Trubion, Wyeth), ofatumumab (HuMax-CD20; Genmab); AME-133 (Eli Lilly), Immu-106 (Immunomedics), all referenced in the background section.

The term “inadequate response to a CD20 antagonist” refers to an inadequate response to previous or current treatment with a CD20 antagonist because of toxicity and/or inadequate efficacy. The inadequate response can be assessed by a clinician skilled in treating the disease in question. A mammal, such as human, who experiences “toxicity” from previous or current treatment with the CD20 antagonist experiences one or more negative side-effects associated therewith such as cardiac, pulmonary, renal side-effects, especially severe side effects, such as acute respiratory distress syndrome, myocardial infarction, ventricular fibrillation, cardiogenic shock, hypoxia, pulmonary infiltrates, acute renal failure, mococutaneous reactions, or infections, such as viral infections. A mammal who experiences “inadequate efficacy” continues to have active disease following previous or current treatment with a CD20 antagonist. For instance, the patient may have active disease activity after 1 month or 3 months of therapy with the CD20 antagonist.

The terms “autoimmune disease” and “autoimmune disorder” are used interchangeably, and refer to a disease or disorder arising from and directed against an individual's own tissues or a co-segregate or manifestation thereof or resulting condition therefrom. “Autoimmune disease” can be an organ-specific disease (i.e., the immune response is specifically directed against an organ system such as the endocrine system, the hematopoietic system, the skin, the cardiopulmonary system, the gastrointestinal and liver systems, the renal system, the thyroid, the ears, the neuromuscular system, the central nervous system, etc.) or a systemic disease which can affect multiple organ systems (for example, systemic lupus erythematosus (SLE), rheumatoid arthritis, polymyositis, etc.). Examples of autoimmune diseases or disorders include, but are not limited to autoimmune rheumatologic disorders, such as arthritis (rheumatoid arthritis such as acute arthritis, chronic rheumatoid arthritis, gouty arthritis, acute gouty arthritis, chronic inflammatory arthritis, degenerative arthritis, infectious arthritis, Lyme arthritis, proliferative arthritis, psoriatic arthritis, vertebral arthritis, and juvenile-onset rheumatoid arthritis, osteoarthritis, arthritis chronica progrediente, arthritis deformans, polyarthritis chronica primaria, reactive arthritis, and ankylosing spondylitis), inflammatory hyperproliferative skin diseases, psoriasis such as plaque psoriasis, gutatte psoriasis, pustular psoriasis, and psoriasis of the nails, atopy including atopic diseases such as hay fever and Job's syndrome, dermatitis including contact dermatitis, chronic contact dermatitis, allergic dermatitis, allergic contact dermatitis, dermatitis herpetiformis, and atopic dermatitis, x-linked hyper IgM syndrome, urticaria such as chronic allergic urticaria and chronic idiopathic urticaria, including chronic autoimmune urticaria, polymyositis/dermatomyositis, juvenile dermatomyositis, toxic epidermal necrolysis, scleroderma (including systemic scleroderma), sclerosis such as systemic sclerosis, multiple sclerosis (MS) such as spino-optical MS, primary progressive MS (PPMS), and relapsing remitting MS (RRMS), progressive systemic sclerosis, atherosclerosis, arteriosclerosis, sclerosis disseminata, and ataxic sclerosis, inflammatory bowel disease (IBD) (for example, Crohn's disease, autoimmune-mediated gastrointestinal diseases, colitis such as ulcerative colitis, colitis ulcerosa, microscopic colitis, collagenous colitis, colitis polyposa, necrotizing enterocolitis, and transmural colitis, and autoimmune inflammatory bowel disease), pyoderma gangrenosum, erythema nodosum, primary sclerosing cholangitis, episcleritis), respiratory distress syndrome, including adult or acute respiratory distress syndrome (ARDS), meningitis, inflammation of all or part of the uvea, iritis, choroiditis, an autoimmune hematological disorder, rheumatoid spondylitis, sudden hearing loss, IgE-mediated diseases such as anaphylaxis and allergic and atopic rhinitis, encephalitis such as Rasmussen's encephalitis and limbic and/or brainstem encephalitis, uveitis, such as anterior uveitis, acute anterior uveitis, granulomatous uveitis, nongranulomatous uveitis, phacoantigenic uveitis, posterior uveitis, or autoimmune uveitis, glomerulonephritis (GN) with and without nephrotic syndrome such as chronic or acute glomerulonephritis such as primary GN, immune-mediated GN, membranous GN (membranous nephropathy), idiopathic membranous GN or idiopathic membranous nephropathy, membrano- or membranous proliferative GN (MPGN), including Type I and Type II, and rapidly progressive GN, allergic conditions and responses, allergic reaction, eczema including allergic or atopic eczema, asthma such as asthma bronchiale, bronchial asthma, and auto-immune asthma, conditions involving infiltration of T cells and chronic inflammatory responses, immune reactions against foreign antigens such as fetal A-B-O blood groups during pregnancy, chronic pulmonary inflammatory disease, autoimmune myocarditis, leukocyte adhesion deficiency, systemic lupus erythematosus (SLE) or systemic lupus erythematodes such as cutaneous SLE, subacute cutaneous lupus erythematosus, neonatal lupus syndrome (NLE), lupus erythematosus disseminatus, lupus (including nephritis, cerebritis, pediatric, non-renal, extra-renal, discoid, alopecia), juvenile onset (Type I) diabetes mellitus, including pediatric insulin-dependent diabetes mellitus (IDDM), adult onset diabetes mellitus (Type II diabetes), autoimmune diabetes, idiopathic diabetes insipidus, immune responses associated with acute and delayed hypersensitivity mediated by cytokines and T-lymphocytes, tuberculosis, sarcoidosis, granulomatosis including lymphomatoid granulomatosis, Wegener's granulomatosis, agranulocytosis, vasculitides including vasculitis (including large vessel vasculitis (including polymyalgia rheumatica and giant cell (Takayasu's) arteritis), medium vessel vasculitis (including Kawasaki's disease and polyarteritis nodosa/periarteritis nodosa), microscopic polyarteritis, CNS vasculitis, necrotizing, cutaneous, or hypersensitivity vasculitis, systemic necrotizing vasculitis, ANCA-associated vasculitis (AAV) such as Churg-Strauss vasculitis or syndrome (CSS)), ANCA-negative vasculitis, temporal arteritis, aplastic anemia, autoimmune aplastic anemia, Coombs positive anemia, Diamond Blackfan anemia, hemolytic anemia or immune hemolytic anemia including autoimmune hemolytic anemia (AIHA), pernicious anemia (anemia perniciosa), Addison's disease, pure red cell anemia or aplasia (PRCA), Factor VIII deficiency, hemophilia A, autoimmune neutropenia, pancytopenia, leukopenia, diseases involving leukocyte diapedesis, CNS inflammatory disorders, multiple organ injury syndrome such as those secondary to septicemia, trauma or hemorrhage, antigen-antibody complex-mediated diseases, anti-glomerular basement membrane disease, anti-phospholipid antibody syndrome, allergic neuritis, Bechet's or Behcet's disease, Castleman's syndrome, Goodpasture's syndrome, Reynaud's syndrome, Sjogren's syndrome, Stevens-Johnson syndrome, pemphigoid such as pemphigoid bullous and skin pemphigoid, pemphigus (including pemphigus vulgaris, pemphigus foliaceus, pemphigus mucus-membrane pemphigoid, and pemphigus erythematosus), autoimmune polyendocrinopathies, Reiter's disease or syndrome, immune complex nephritis, antibody-mediated nephritis, neuromyelitis optica (NMO; also known as Devic's syndrome), polyneuropathies, chronic neuropathy such as IgM polyneuropathies or IgM-mediated neuropathy, thrombocytopenia (as developed by myocardial infarction patients, for example), including thrombotic thrombocytopenic purpura (TTP), post-transfusion purpura (PTP), heparin-induced thrombocytopenia, and autoimmune or immune-mediated thrombocytopenia such as idiopathic thrombocytopenic purpura (ITP) including chronic or acute ITP, autoimmune disease of the testis and ovary including autoimune orchitis and oophoritis, primary hypothyroidism, hypoparathyroidism, autoimmune endocrine diseases including thyroiditis such as autoimmune thyroiditis, Hashimoto's disease, chronic thyroiditis (Hashimoto's thyroiditis), or subacute thyroiditis, autoimmune thyroid disease, idiopathic hypothyroidism, Grave's disease, polyglandular syndromes such as autoimmune polyglandular syndromes (or polyglandular endocrinopathy syndromes), paraneoplastic syndromes, including neurologic paraneoplastic syndromes such as Lambert-Eaton myasthenic syndrome or Eaton-Lambert syndrome, stiff-man or stiff-person syndrome, encephalomyelitis such as allergic encephalomyelitis or encephalomyelitis allergica and experimental allergic encephalomyelitis (EAE), myasthenia gravis such as thymoma-associated myasthenia gravis, cerebellar degeneration, neuromyotonia, opsoclonus or opsoclonus myoclonus syndrome (OMS), and sensory neuropathy, multifocal motor neuropathy, Sheehan's syndrome, autoimmune hepatitis, chronic hepatitis, lupoid hepatitis, giant cell hepatitis, chronic active hepatitis or autoimmune chronic active hepatitis, lymphoid interstitial pneumonitis (LIP), bronchiolitis obliterans (non-transplant) vs NSIP, Guillain-Barre syndrome, Berger's disease (IgA nephropathy), idiopathic IgA nephropathy, linear IgA dermatosis, primary biliary cirrhosis, pneumonocirrhosis, autoimmune enteropathy syndrome, Celiac disease, Coeliac disease, celiac sprue (gluten enteropathy), refractory sprue, idiopathic sprue, cryoglobulinemia, amylotrophic lateral sclerosis (ALS; Lou Gehrig's disease), coronary artery disease, autoimmune ear disease such as autoimmune inner ear disease (AIED), autoimmune hearing loss, opsoclonus myoclonus syndrome (OMS), polychondritis such as refractory or relapsed polychondritis, pulmonary alveolar proteinosis, amyloidosis, scleritis, a non-cancerous lymphocytosis, a primary lymphocytosis, which includes monoclonal B cell lymphocytosis (e.g., benign monoclonal gammopathy and monoclonal garnmopathy of undetermined significance, MGUS), peripheral neuropathy, paraneoplastic syndrome, channelopathies such as epilepsy, migraine, arrhythmia, muscular disorders, deafness, blindness, periodic paralysis, and channelopathies of the CNS, autism, inflammatory myopathy, focal segmental glomerulosclerosis (FSGS), endocrine opthalmopathy, uveoretinitis, chorioretinitis, autoimmune hepatological disorder, fibromyalgia, multiple endocrine failure, Schmidt's syndrome, adrenalitis, gastric atrophy, presenile dementia, demyelinating diseases such as autoimmune demyelinating diseases and chronic inflammatory demyelinating polyneuropathy, diabetic nephropathy, Dressler's syndrome, alopecia greata, CREST syndrome (calcinosis, Raynaud's phenomenon, esophageal dysmotility, sclerodactyl), and telangiectasia), male and female autoimmune infertility, mixed connective tissue disease, Chagas' disease, rheumatic fever, recurrent abortion, farmer's lung, erythema multiforme, post-cardiotomy syndrome, Cushing's syndrome, bird-fancier's lung, allergic granulomatous angiitis, benign lymphocytic angiitis, Alport's syndrome, alveolitis such as allergic alveolitis and fibrosing alveolitis, interstitial lung disease, transfusion reaction, leprosy, malaria, leishmaniasis, kypanosomiasis, schistosomiasis, ascariasis, aspergillosis, Sampter's syndrome, Caplan's syndrome, dengue, endocarditis, endomyocardial fibrosis, diffuse interstitial pulmonary fibrosis, interstitial lung fibrosis, pulmonary fibrosis, idiopathic pulmonary fibrosis, cystic fibrosis, endophthalmitis, erythema elevatum et diutinum, erythroblastosis fetalis, eosinophilic faciitis, Shulman's syndrome, Felty's syndrome, flariasis, cyclitis such as chronic cyclitis, heterochronic cyclitis, iridocyclitis (acute or chronic), or Fuch's cyclitis, Henoch-Schonlein purpura, human immunodeficiency virus (HIV) infection, echovirus infection, cardiomyopathy, Alzheimer's disease, parvovirus infection, rubella virus infection, post-vaccination syndromes, congenital rubella infection, Epstein-Barr virus infection, mumps, Evan's syndrome, autoimmune gonadal failure, Sydenham's chorea, post-streptococcal nephritis, thromboangitis ubiterans, thyrotoxicosis, tabes dorsalis, chorioiditis, giant cell polymyalgia, endocrine ophthamopathy, chronic hypersensitivity pneumonitis, keratoconjunctivitis sicca, epidemic keratoconjunctivitis, idiopathic nephritic syndrome, minimal change nephropathy, benign familial and ischemia-reperfusion injury, retinal autoimmunity, joint inflammation, bronchitis, chronic obstructive airway disease, silicosis, aphthae, aphthous stomatitis, arteriosclerotic disorders, aspermiogenese, autoimmune hemolysis, Boeck's disease, cryoglobulinemia, Dupuytren's contracture, endophthalmia phacoanaphylactica, enteritis allergica, erythema nodosum leprosum, idiopathic facial paralysis, chronic fatigue syndrome, febris rheumatica, Hamman-Rich's disease, sensoneural hearing loss, haemoglobinuria paroxysmatica, hypogonadism, ileitis regionalis, leucopenia, mononucleosis infectiosa, traverse myelitis, primary idiopathic myxedema, nephrosis, ophthalmia symphatica, orchitis granulomatosa, pancreatitis, polyradiculitis acuta, pyoderma gangrenosum, Quervain's thyreoiditis, acquired spenic atrophy, infertility due to antispermatozoan antobodies, non-malignant thymoma, vitiligo, SCID and Epstein-Barr virus-associated diseases, acquired immune deficiency syndrome (AIDS), parasitic diseases such as Lesihmania, toxic-shock syndrome, food poisoning, conditions involving infiltration of T cells, leukocyte-adhesion deficiency, immune responses associated with acute and delayed hypersensitivity mediated by cytokines and T-lymphocytes, diseases involving leukocyte diapedesis, multiple organ injury syndrome, antigen-antibody complex-mediated diseases, antiglomerular basement membrane disease, allergic neuritis, autoimmune polyendocrinopathies, oophoritis, primary myxedema, autoimmune atrophic gastritis, sympathetic ophthalmia, rheumatic diseases, mixed connective tissue disease, nephrotic syndrome, insulitis, polyendocrine failure, peripheral neuropathy, autoimmune polyglandular syndrome type I, adult-onset idiopathic hypoparathyroidism (AOIH), alopecia totalis, dilated cardiomyopathy, epidermolisis bullosa acquisita (EBA), hemochromatosis, myocarditis, nephrotic syndrome, primary sclerosing cholangitis, purulent or nonpurulent sinusitis, acute or chronic sinusitis, ethmoid, frontal, maxillary, or sphenoid sinusitis, an eosinophil-related disorder such as eosinophilia, pulmonary infiltration eosinophilia, eosinophilia-myalgia syndrome, Loffler's syndrome, chronic eosinophilic pneumonia, tropical pulmonary eosinophilia, bronchopneumonic aspergillosis, aspergilloma, or granulomas containing eosinophils, anaphylaxis, seronegative spondyloarthritides, polyendocrine autoimmune disease, sclerosing cholangitis, sclera, episclera, chronic mucocutaneous candidiasis, Bruton's syndrome, transient hypogammaglobulinemia of infancy, Wiskott-Aldrich syndrome, ataxia telangiectasia, autoimmune disorders associated with collagen disease, rheumatism, neurological disease, lymphadenitis, ischemic re-perfusion disorder, reduction in blood pressure response, vascular dysfunction, antgiectasis, tissue injury, cardiovascular ischemia, hyperalgesia, cerebral ischemia, and disease accompanying vascularization, allergic hypersensitivity disorders, glomerulonephritides, reperfusion injury, reperfusion injury of myocardial or other tissues, dermatoses with acute inflammatory components, acute purulent meningitis or other central nervous system inflammatory disorders, ocular and orbital inflammatory disorders, granulocyte transfusion-associated syndromes, cytokine-induced toxicity, acute serious inflammation, chronic intractable inflammation, pyelitis, pneumonocirrhosis, diabetic retinopathy, diabetic large-artery disorder, endarterial hyperplasia, peptic ulcer, valvulitis, and endometriosis.

The term “rheumatoid arthritis” or “RA” is used herein in the broadest sense and refers to a recognized disease state which may be diagnosed according to the 2000 revised American Rheumatoid Association criteria for the classification of rheumatoid arthritis, or any similar criteria. Physiological indicators of RA include, symmetric joint swelling which is characteristic though not invariable in rheumatoid arthritis. Fusiform swelling of the proximal interphalangeal (PIP) joints of the hands as well as metacarpophalangeal (MCP), wrists, elbows, knees, ankles and metatarsophalangeal (MTP) joints are commonly affected and swelling is easily detected. Pain on passive motion is the most sensitive test for joint inflammation, and inflammation and structural deformity often limits the range of motion for the affected joint. Typical visible changes include ulnar deviation of the fingers at the MCP joints, hyperextension or hyperflexion of the MCP and PIP joints, flexion contractures of the elbows, and subluxation of the carpal bones and toes. The subject with rheumatoid arthritis may be resistant to DMARDs, in that the DMARDs are not effective or fully effective in treating symptoms. Rheumatoid arthritis can be described as mild, moderate, or severe. Mild RA is characterized by the involvement of at least 3 joints (simultaneously) with no extra-articular disease. The rheumatoid factor (RF) is typically negative and there are no erosions on the radiographs. Moderate disease includes active inflammation of 6-20 joints and usually involves radiograph changes and a positive RF—there are typically no extra-articular manifestations. Moderate disease suggests the need for an NSAID and DMARD together, as opposed to simply NSAID therapy. Severe RA is characterized by the involvement of more than 20 joints, includes positive RF (usually high titer) and extra-articular disease, and typically additionally causes other systemic effects including hypoalbuminemia and anemia of chronic disease. There is rapidly decreasing functional capacity in the severe form of RA. The term “rheumatoid arthritis” or “RA,” as used herein, includes all forms and stages of RA, including, without limitation, active rheumatoid arthritis, early and late RA based on how long the patient has had RA, and moderate to severe rheumatoid arthritis.

Rheumatoid factor (RF) is an immunoglobulin directed against the Fc portion of another immunoglobulin commonly used as a blood test for the diagnosis of rheumatoid arthritis. It can self-aggregate into a lattice-like form within joint cavities to provide a surface onto which inflammatory cells can adhere and act. Rheumatoid arthritis patients with a high titer of RF (approximately 80% of patients) have more aggressive disease, with a worse long-term outcome and increased mortality over those who are RF negative.

A patient with “active rheumatoid arthritis” means a patient with active and not latent symptoms of rheumatoid arthritis. For the purpose of the present invention “active rheumatoid arthritis” is defined by the presence of at least 4 swollen joints.

Subjects with “early active rheumatoid arthritis” are those subjects with active rheumatoid arthritis diagnosed for at least 8 weeks but no longer than four years, according to the revised 1987 ACR criteria for the classification of RA.

“Treating” or “treatment” or “alleviation” refers to therapeutic treatment and prophylactic or preventative measures, wherein the object is to prevent or slow down (lessen) the targeted pathologic condition or disorder. A subject is successfully “treated” for an autoimmune disease if, after receiving a therapeutic amount of a CD20 antagonist, such as a CD20 binding antibody of the invention according to the methods of the present invention, the subject shows observable and/or measurable reduction in or absence of one or more signs and symptoms of the particular autoimmune disease. For example, if the target disease is rheumatoid arthritis, those in need of treatment include those already with the disease as well as those in which the development of the disease, including various characteristics of the disease, such as joint damage or the progress of joint damage, is to be prevented. Hence, the subject may have been diagnosed as having rheumatoid arthritis or may be predisposed or susceptible to rheumatoid arthritis, or may have mild or moderate rheumatoid arthritis, which is likely to progress in the absence of treatment. Treatment is successful if the disease and/or various characteristics or symptoms of the disease, is/are alleviated or healed, or progression of the disease is halted or slowed down as compared to prior to administration. Successful treatment further includes complete or partial prevention of the development of the disease. For purposes herein, slowing down or reducing the disease or symptoms of the disease or the progression of symptoms of the disease is the same as arrest, decrease, or reversal in the symptoms. Symptoms of rheumatoid arthritis, without limitation, include any of the symptoms listed in the 2000 revised American Rheumatoid Association criteria for the classification of rheumatoid arthritis, such as, for example, joint swelling, joint damage, bone deformity, bone erosion, pain, inflammation, and systemic effects associated with more advance forms of rheumatoid arthritis, including hypoalbuminemia and anemia of chronic disease.

The phrase “effective amount” is used herein to refer to therapeutically and preventatively effective amount. Thus, in the present methods of the invention, the “effective amount” refers to an amount of a CD20 antibody or another CD20 antagonist that is effective for treating (including prevention) of rheumatoid arthritis. As a result of administering an “effective amount” the target disease and/or various characteristics or symptoms of the disease, is/are alleviated or healed, or progression of the disease is halted or slowed down as compared to prior to administration. Successful treatment further includes complete or partial prevention of the development of the disease. For purposes herein, slowing down or reducing the disease or symptoms of the disease or the progression of symptoms of the disease is the same as arrest, decrease, or reversal in the symptoms. Symptoms of rheumatoid arthritis, without limitation, include any of the symptoms listed in the 2000 revised American Rheumatoid Association criteria for the classification of rheumatoid arthritis, such as, for example, joint swelling, joint damage, bone deformity, bone erosion, pain, inflammation, and systemic effects associated with more advance forms of rheumatoid arthritis, including hypoalbuminemia and anemia of chronic disease.

The phrase “complete therapeutically effective amount” administered as a “single intravenous (i.v.) infusion” is used herein to mean that the total amount of a CD20 antagonist, such as a CD20 antibody, that is effective in the therapeutic treatment of a target disease, such as rheumatoid arthritis, is administered as a single i.v. infusion, instead of administration of two or more partial dosages (smaller than the therapeutically effective amount) consecutively, in separate infusions such as several days or weeks apart.

The “CD20” antigen is a non-glycosylated, transmembrane phosphoprotein with a molecular weight of approximately 35 kD that is found on the surface of greater than 90% of B cells from peripheral blood or lymphoid organs. CD20 is expressed during early pre-B cell development and remains until plasma cell differentiation; it is not found on human stem cells, lymphoid progenitor cells or normal plasma cells. CD20 is present on both normal B cells as well as malignant B cells. Other names for CD20 in the literature include “B-lymphocyte-restricted differentiation antigen” and “Bp35”. The CD20 antigen is described in, for example, Clark and Ledbetter, Adv. Can. Res. 52:81-149 (1989) and Valentine et al. J. Biol. Chem. 264(19):11282-11287 (1989).

The term “antibody” is used in the broadest sense and specifically covers monoclonal antibodies (including full length monoclonal antibodies), multispecific antibodies (e.g., bispecific antibodies), single chain antibodies and antibody fragments so long as they exhibit the desired biological activity or function.

The biological activity of the CD20 binding antibodies of the invention will include binding of the antibody to human CD20, more preferably binding to human and other primate CD20 (including cynomolgus monkey, rhesus monkey, chimpanzees, baboons). The antibodies will bind CD20 with a K_{d} value of no higher than 1×10⁻⁸, preferably a K_{d} value no higher than about 1×10⁻⁹, and be able to kill or deplete B cells in vivo, preferably by at least 20% when compared to the appropriate negative control which is not treated with such an antibody. B cell depletion can be a result of one or more of ADCC, CDC, apoptosis, or other mechanism. In some embodiments of disease treatment herein, specific effector functions or mechanisms may be desired over others and certain variants of the humanized 2H7 or certain human CD20 binding antibodies are preferred to achieve those biological functions, such as ADCC.

“Antibody fragments” comprise a portion of a full length antibody, generally the antigen binding or variable region thereof. Examples of antibody fragments include Fab, Fab′, F(ab′)₂, and Fv fragments; diabodies; linear antibodies; single-chain antibody molecules; and multispecific antibodies formed from antibody fragments. “Fv” is the minimum antibody fragment which contains a complete antigen-recognition and -binding site. This fragment consists of a dimer of one heavy- and one light-chain variable region domain in tight, non-covalent association. From the folding of these two domains emanate six hypervariable loops (3 loops each from the H and L chain) that contribute the amino acid residues for antigen binding and confer antigen binding specificity to the antibody. However, even a single variable domain (or half of an Fv comprising only three CDRs specific for an antigen) has the ability to recognize and bind antigen, although at a lower affinity than the entire binding site.

The term “monoclonal antibody” as used herein refers to an antibody from a population of substantially homogeneous antibodies, i.e., the individual antibodies comprising the population are identical and/or bind the same epitope(s), except for possible variants that may arise during production of the monoclonal antibody, such variants generally being present in minor amounts. Such monoclonal antibody typically includes an antibody comprising a polypeptide sequence that binds a target, wherein the target-binding polypeptide sequence was obtained by a process that includes the selection of a single target binding polypeptide sequence from a plurality of polypeptide sequences. For example, the selection process can be the selection of a unique clone from a plurality of clones, such as a pool of hybridoma clones, phage clones or recombinant DNA clones. It should be understood that the selected target binding sequence can be further altered, for example, to improve affinity for the target, to humanize the target binding sequence, to improve its production in cell culture, to reduce its immunogenicity in vivo, to create a multispecific antibody, etc., and that an antibody comprising the altered target binding sequence is also a monoclonal antibody of this invention. In contrast to polyclonal antibody preparations which typically include different antibodies directed against different determinants (epitopes), each monoclonal antibody of a monoclonal antibody preparation is directed against a single determinant on an antigen. In addition to their specificity, the monoclonal antibody preparations are advantageous in that they are typically uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including, for example, the hybridoma method (e.g., Kohler et al., Nature, 256:495 (1975); Harlow et al., Antibodies: A Laboratory Manual, (Cold Spring Harbor Laboratory Press, 2nd ed. 1988); Hammerling et al., in: Monoclonal Antibodies and T-Cell Hybridomas 563-681, (Elsevier, N.Y., 1981)), recombinant DNA methods (see, e.g., U.S. Pat. No. 4,816,567), phage display technologies (see, e.g., Clackson et al., Nature, 352:624-628 (1991); Marks et al., J. Mol. Biol., 222:581-597 (1991); Sidhu et al., J. Mol. Biol. 338(2):299-310 (2004); Lee et al., J. Mol. Biol. 340(5):1073-1093 (2004); Fellouse, Proc. Nat. Acad. Sci. USA 101(34):12467-12472 (2004); and Lee et al. J. Immunol. Methods 284(1-2):119-132 (2004), and technologies for producing human or human-like antibodies in animals that have parts or all of the human immunoglobulin loci or genes encoding human immunoglobulin sequences (see, e.g., WO 1998/24893; WO 1996/34096; WO 1996/33735; WO 1991/10741; Jakobovits et al., Proc. Natl. Acad. Sci. USA, 90:2551 (1993); Jakobovits et al., Nature, 362:255-258 (1993); Bruggemann et al., Year in Immuno., 7:33 (1993); U.S. Pat. Nos. 5,545,806; 5,569,825; 5,591,669 (all of GenPharm); 5,545,807; WO 1997/17852; U.S. Pat. Nos. 5,545,807; 5,545,806; 5,569,825; 5,625,126; 5,633,425; and 5,661,016; Marks et al., Bio/Technology, 10: 779-783 (1992); Lonberg et al., Nature, 368: 856-859 (1994); Morrison, Nature, 368: 812-813 (1994); Fishwild et al., Nature Biotechnology, 14: 845-851 (1996); Neuberger, Nature Biotechnology, 14: 826 (1996); and Lonberg and Huszar, Intern. Rev. Immunol., 13: 65-93 (1995).

“Functional fragments” of the CD20 binding antibodies of the invention are those fragments that retain binding to CD20 with substantially the same affinity as the intact full length molecule from which they are derived and show biological activity including depleting B cells as measured by in vitro or in vivo assays such as those described herein.

The term “variable” refers to the fact that certain segments of the variable domains differ extensively in sequence among antibodies. The V domain mediates antigen binding and define specificity of a particular antibody for its particular antigen. However, the variability is not evenly distributed across the 110-amino acid span of the variable domains. Instead, the V regions consist of relatively invariant stretches called framework regions (FRs) of 15-30 amino acids separated by shorter regions of extreme variability called “hypervariable regions” that are each 9-12 amino acids long. The variable domains of native heavy and light chains each comprise four FRs, largely adopting a β-sheet configuration, connected by three hypervariable regions, which form loops connecting, and in some cases forming part of, the β-sheet structure. The hypervariable regions in each chain are held together in close proximity by the FRs and, with the hypervariable regions from the other chain, contribute to the formation of the antigen-binding site of antibodies (see Kabat et al., Sequences of Proteins of Immunological Interest, 5th Ed. Public Health Service, National Institutes of Health, Bethesda, Md. (1991)). The constant domains are not involved directly in binding an antibody to an antigen, but exhibit various effector functions, such as participation of the antibody in antibody dependent cellular cytotoxicity (ADCC).

The term “hypervariable region” when used herein refers to the amino acid residues of an antibody which are responsible for antigen-binding. The hypervariable region generally comprises amino acid residues from a “complementarity determining region” or “CDR” (e.g. around about residues 24-34 (L1), 50-56 (L2) and 89-97 (L3) in the V_{L}, and around about 31-35B (H1), 50-65 (H2) and 95-102 (H3) in the V_{H} (Kabat et al., Sequences of Proteins of Immunological Interest, 5th Ed. Public Health Service, National Institutes of Health, Bethesda, Md. (1991)) and/or those residues from a “hypervariable loop” (e.g. residues 26-32 (L1), 50-52 (L2) and 91-96 (L3) in the V_{L}, and 26-32 (H1), 52A-55 (H2) and 96-101 (H3) in the V_{H} (Chothia and Lesk J. Mol. Biol. 196:901-917 (1987)).

As referred to herein, the “consensus sequence” or consensus V domain sequence is an artificial sequence derived from a comparison of the amino acid sequences of known human immunoglobulin variable region sequences. Based on these comparisons, recombinant nucleic acid sequences encoding the V domain amino acids that are a consensus of the sequences derived from the human κ and the human H chain subgroup III V domains were prepared. The consensus V sequence does not have any known antibody binding specificity or affinity.

“Chimeric” antibodies (immunoglobulins) have a portion of the heavy and/or light chain identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al., Proc. Natl. Acad. Sci. USA 81:6851-6855 (1984)). Humanized antibody as used herein is a subset of chimeric antibodies.

“Humanized” forms of non-human (e.g., murine) antibodies are chimeric antibodies which contain minimal sequence derived from non-human immunoglobulin. For the most part, humanized antibodies are human immunoglobulins (recipient or acceptor antibody) in which hypervariable region residues of the recipient are replaced by hypervariable region residues from a non-human species (donor antibody) such as mouse, rat, rabbit or nonhuman primate having the desired specificity, affinity, and capacity. In some instances, Fv framework region (FR) residues of the human immunoglobulin are replaced by corresponding non-human residues. Furthermore, humanized antibodies may comprise residues which are not found in the recipient antibody or in the donor antibody. These modifications are made to further refine antibody performance such as binding affinity. Generally, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence although the FR regions may include one or more amino acid substitutions that improve binding affinity. The number of these amino acid substitutions in the FR are typically no more than 6 in the H chain, and in the L chain, no more than 3. The humanized antibody optionally also will comprise at least a portion of an immunoglobulin constant region (Fc), typically that of a human immunoglobulin. For further details, see Jones et al., Nature 321:522-525 (1986); Reichmann et al., Nature 332:323-329 (1988); and Presta, Curr. Op. Struct. Biol. 2:593-596 (1992).

Antibody “effector functions” refer to those biological activities attributable to the Fc region (a native sequence Fc region or amino acid sequence variant Fc region) of an antibody, and vary with the antibody isotype. Examples of antibody effector functions include: C1q binding and complement dependent cytotoxicity; Fc receptor binding; antibody-dependent cell-mediated cytotoxicity (ADCC); phagocytosis; down regulation of cell surface receptors (e.g. B cell receptor); and B cell activation.

“Antibody-dependent cell-mediated cytotoxicity” or “ADCC” refers to a form of cytotoxicity in which secreted Ig bound onto Fc receptors (FcRs) present on certain cytotoxic cells (e.g. Natural Killer (NK) cells, neutrophils, and macrophages) enable these cytotoxic effector cells to bind specifically to an antigen-bearing target cell and subsequently kill the target cell with cytotoxins. The antibodies “arm” the cytotoxic cells and are absolutely required for such killing. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol 9:457-92 (1991). To assess ADCC activity of a molecule of interest, an in vitro ADCC assay, such as that described in U.S. Pat. No. 5,500,362 or 5,821,337 may be performed. Useful effector cells for such assays include peripheral blood mononuclear cells (PBMC) and Natural Killer (NK) cells. Alternatively, or additionally, ADCC activity of the molecule of interest may be assessed in vivo, e.g., in a animal model such as that disclosed in Clynes et al. PNAS (USA) 95:652-656 (1998).

“Fc receptor” or “FcR” describes a receptor that binds to the Fc region of an antibody. The preferred FcR is a native sequence human FcR. Moreover, a preferred FcR is one which binds an IgG antibody (a gamma receptor) and includes receptors of the FcγRI, FcγRII, and FcγRIII subclasses, including allelic variants and alternatively spliced forms of these receptors. FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain. (see review M. in Daëron, Annu. Rev. Immunol. 15:203-234 (1997)). FcRs are reviewed in Ravetch and Kinet, Annu. Rev. Immunol 9:457-92 (1991); Capel et al., Immunomethods 4:25-34 (1994); and de Haas et al., J. Lab. Clin. Med. 126:330-41 (1995). Other FcRs, including those to be identified in the future, are encompassed by the term “FcR” herein. The term also includes the neonatal receptor, FcRn, which is responsible for the transfer of maternal IgGs to the fetus (Guyer et al., J. Immunol. 117:587 (1976) and Kim et al., J. Immunol. 24:249 (1994)).

WO00/42072 (Presta) and WO 2004/056312 (Lowman et al) describe antibody variants with improved or diminished binding to FcRs. The content of these patent publications are specifically incorporated herein by reference. See, also, Shields et al., Journal of Biological Chemistry 276(9):6591-6604 (2001).

“Complement dependent cytotoxicity” or “CDC” refers to the lysis of a target cell in the presence of complement. Activation of the classical complement pathway is initiated by the binding of the first component of the complement system (C1q) to antibodies (of the appropriate subclass) which are bound to their cognate antigen. To assess complement activation, a CDC assay, e.g. as described in Gazzano-Santoro et al., J. Immunol. Methods 202:163 (1996), may be performed.

Polypeptide variants with altered Fc region amino acid sequences and increased or decreased Clq binding capability are described in U.S. Pat. No. 6,194,551B1, WO99/51642. The contents of those patent publications are specifically incorporated herein by reference. See, also, Idusogie et al. J. Immunol. 164: 4178-4184 (2000).

A “naked antibody” is an antibody (as herein defined) that is not conjugated to a heterologous molecule, such as a cytotoxic moiety, polymer, or radiolabel.

An “isolated” antibody is one that has been identified and separated and/or recovered from a component of its natural environment. Contaminant components of its natural environment are materials that would interfere with diagnostic or therapeutic uses for the antibody, and may include enzymes, hormones, and other proteinaceous or non-proteinaceous solutes. In preferred embodiments, the antibody will be purified (1) to greater than 95% by weight of antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or, preferably, silver stain. Isolated antibody includes the antibody in situ within recombinant cells since at least one component of the antibody's natural environment will not be present. Ordinarily, however, isolated antibody will be prepared by at least one purification step.

“Joint damage” is used in the broadest sense and refers to damage or partial or complete destruction to any part of one or more joints, including the connective tissue and cartilage, where damage includes structural and/or functional damage of any cause, and may or may not cause joint pain/arthalgia. It specifically includes, without limitation, joint damage associated with or resulting from inflammatory joint disease as well as non-inflammatory joint disease. This damage may be caused, for example, an autoimmune disease such as lupus (e.g., systemic lupus erythematosus), arthritis (e.g., acute and chronic arthritis, rheumatoid arthritis including juvenile-onset rheumatoid arthritis, juvenile idiopathic arthritis (JIA), or juvenile RA (JRA), and stages such as rheumatoid synovitis, gout or gouty arthritis, acute immunological arthritis, chronic inflammatory arthritis, degenerative arthritis, type II collagen-induced arthritis, infectious arthritis, septic arthritis, Lyme arthritis, proliferative arthritis, psoriatic arthritis, Still's disease, vertebral arthritis, osteoarthritis, arthritis chronica progrediente, arthritis deformans, polyarthritis chronica primaria, reactive arthritis, menopausal arthritis, estrogen-depletion arthritis, and ankylosing spondylitis/rheumatoid spondylitis), rheumatic autoimmune disease other than RA, significant systemic involvement secondary to RA (including but not limited to vasculitis, pulmonary fibrosis or Felty's syndrome), Sjögren's syndrome, particular secondary such syndrome, and secondary limited cutaneous vasculitis with RA.

The term “immunosuppressive agent” as used herein for adjunct therapy refers to substances that act to suppress or mask the immune system of the mammal being treated herein. This would include substances that suppress cytokine production, down-regulate or suppress self-antigen expression, or mask the MHC antigens. Examples of such agents include 2-amino-6-aryl-5-substituted pyrimidines (see U.S. Pat. No. 4,665,077); non-steroidal anti-inflammatory drugs (NSAIDs); ganciclovir, tacrolimus, glucocorticoids such as cortisol or aldosterone, anti-inflammatory agents such as a cyclooxygenase inhibitor, a 5-lipoxygenase inhibitor, or a leukotriene receptor antagonist; purine antagonists such as azathioprine or mycophenolate mofetil (MMF); alkylating agents such as cyclophosphamide; bromocryptine; danazol; dapsone; glutaraldehyde (which masks the MHC antigens, as described in U.S. Pat. No. 4,120,649); anti-idiotypic antibodies for MHC antigens and MHC fragments; cyclosporin A; steroids such as corticosteroids or glucocorticosteroids or glucocorticoid analogs, e.g., prednisone, methylprednisolone, including SOLU-MEDROLO methylprednisolone sodium succinate, and dexamethasone; dihydrofolate reductase inhibitors such as methotrexate (oral or subcutaneous); anti-malarial agents such as chloroquine and hydroxychloroquine; sulfasalazine; leflunomide; cytokine antagonists such as cytokine antibodies or cytokine receptor antibodies including anti-interferon-alpha, -beta, or -gamma antibodies, anti-tumor necrosis factor (TNF)-alpha antibodies (infliximab (REMICADEO) or adalimumab), anti-TNF-alpha immunoadhesin (etanercept), anti-TNF-beta antibodies, anti-interleukin-2 (IL-2) antibodies and anti-IL-2 receptor antibodies, and anti-interleukin-6 (IL-6) receptor antibodies and antagonists; anti-LFA-1 antibodies, including anti-CD11a and anti-CD18 antibodies; anti-L3T4 antibodies; heterologous anti-lymphocyte globulin; pan-T antibodies, preferably anti-CD3 or anti-CD4/CD4a antibodies; soluble peptide containing a LFA-3 binding domain (WO 90/08187 published Jul. 26, 1990); streptokinase; transforming growth factor-beta (TGF-beta); streptodornase; RNA or DNA from the host; FK506; RS-61443; chlorambucil; deoxyspergualin; rapamycin; T-cell receptor (Cohen et al., U.S. Pat. No. 5,114,721); T-cell receptor fragments (Offner et al., Science, 251: 430-432 (1991); WO 90/11294; Ianeway, Nature, 341: 482 (1989); and WO 91/01133); BAFF antagonists such as BAFF antibodies and BR3 antibodies and zTNF4 antagonists (for review, see Mackay and Mackay, Trends Immunol., 23:113-5 (2002)); biologic agents that interfere with T cell helper signals, such as anti-CD40 receptor or anti-CD40 ligand (CD 154), including blocking antibodies to CD40-CD40 ligand (e.g., Durie et al., Science, 261: 1328-30 (1993); Mohan et al., J. Immunol., 154: 1470-80 (1995)) and CTLA4-Ig (Finck et al., Science, 265: 1225-7 (1994)); and T-cell receptor antibodies (EP 340,109) such as T10B9. Some immunosuppressive agents herein are also DMARDs, such as methotrexate. Examples of preferred immunosuppressive agents herein include cyclophosphamide, chlorambucil, azathioprine, leflunomide, MMF, or methotrexate.

Examples of “disease-modifying anti-rheumatic drugs” or “DMARDs” include hydroxycloroquine, sulfasalazine, methotrexate, leflunomide, etanercept, infliximab (plus oral and subcutaneous methotrexate), azathioprine, D-penicillamine, gold salts (oral), gold salts (intramuscular), minocycline, cyclosporine including cyclosporine A and topical cyclosporine, staphylococcal protein A (Goodyear and Silverman, J. Exp. Med., 197, (9), p1125-39 (2003)), including salts and derivatives thereof, etc. A preferred DMARD herein is methotrexate.

Examples of “non-steroidal anti-inflammatory drugs” or “NSAIDs” include aspirin, acetylsalicylic acid, ibuprofen, flurbiprofen, naproxen, indomethacin, sulindac, tolmetin, phenylbutazone, diclofenac, ketoprofen, benorylate, mefenamic acid, methotrexate, fenbufen, azapropazone; COX-2 inhibitors such as celecoxib (CELEBREX®; 4-(5-(4-methylphenyl)-3-(trifluoromethyl)-1H-pyrazol-1-yl) benzenesulfonamide, valdecoxib (BEXTRA®), meloxicam (MOBIC®), GR 253035 (Glaxo Wellcome); and MK966 (Merck Sharp & Dohme), including salts and derivatives thereof, etc. Preferably, they are aspirin, naproxen, ibuprofen, indomethacin, or tolmetin.

Examples of “integrin antagonists or antibodies” herein include an LFA-1 antibody, such as efalizumab (RAPTIVA®) commercially available from Genentech, or an alpha 4 integrin antibody such as natalizumab (ANTEGREN®) available from Biogen, or diazacyclic phenylalanine derivatives (WO 2003/89410), phenylalanine derivatives (WO 2003/70709, WO 2002/28830, WO 2002/16329 and WO 2003/53926), phenylpropionic acid derivatives (WO 2003/10135), enamine derivatives (WO 2001/79173), propanoic acid derivatives (WO 2000/37444), alkanoic acid derivatives (WO 2000/32575), substituted phenyl derivatives (U.S. Pat. Nos. 6,677,339 and 6,348,463), aromatic amine derivatives (U.S. Pat. No. 6,369,229), ADAM disintegrin domain polypeptides (US2002/0042368), antibodies to alphavbeta3 integrin (EP 633945), aza-bridged bicyclic amino acid derivatives (WO 2002/02556), etc.

“Corticosteroid” refers to any one of several synthetic or naturally occurring substances with the general chemical structure of steroids that mimic or augment the effects of the naturally occurring corticosteroids. Examples of synthetic corticosteroids include prednisone, prednisolone (including methylprednisolone, such as SOLU-MEDROL® methylprednisolone sodium succinate), dexamethasone or dexamethasone triamcinolone, hydrocortisone, and betamethasone. The preferred corticosteroids herein are prednisone, methylprednisolone, hydrocortisone, or dexamethasone.

“TNF” drugs for the treatment of rheumatoid arthritis include molecules that target TNFα ligand, as well as molecules that target the receptors for TNF-TNFα namely TNFR-1 and TNFR-2.

For the purposes herein, “tumor necrosis factor alpha (TNF-alpha)” refers to a human TNF-alpha molecule comprising the amino acid sequence as described in Pennica et al., Nature, 312:721 (1984) or Aggarwal et al., JBC, 260:2345 (1985).

A “TNFα inhibitor” or a “TNFα antagonist” herein is an agent that inhibits, to some extent, a biological function of TNFα, generally through binding to TNFα or to the receptors of TNFα, and neutralizing the activity of TNFα. Examples of TNF inhibitors specifically contemplated herein are Etanercept (ENBREL®)), Infliximab (REMICADE®) and Adalimumab (HUMIRA™).

The term “inadequate response to a TNFa-inhibitor” refers to an inadequate response to previous or current treatment with a TNFa-inhibitor because of toxicity and/or inadequate efficacy. The inadequate response can be assessed by a clinician skilled in treating the disease in question. A mammal who experiences “toxicity” from previous or current treatment with the TNFα-inhibitor experiences one or more negative side-effects associated therewith such as infection (especially serious infections), congestive heart failure, demyelination (leading to multiple sclerosis), hypersensitivity, neurologic events, autoimmunity, non-Hodgkin's lymphoma, tuberculosis (TB), autoantibodies, etc. A mammal who experiences “inadequate efficacy” continues to have active disease following previous or current treatment with a TNFα-inhibitor. For instance, the patient may have active disease activity after 1 month or 3 months of therapy with the TNFa-inhibitor.

A “package insert” is used to refer to instructions customarily included in commercial packages of therapeutic products, that contain information about the indications, usage, dosage, administration, contraindications, other therapeutic products to be combined with the packaged product, and/or warnings concerning the use of such therapeutic products, etc.

A “medicament” is an active drug to treat the joint damage or its symptoms or side effects.

The term “relapse” is used herein the refer to the return of signs and symptoms of a disease after a patient has enjoyed a remission. Thus, if initially the target disease and/or various characteristics or symptoms of the disease, were alleviated or healed, or progression of the disease was halted or slowed down, and subsequently the disease or one or more characteristics of the disease return, the patient is referred to as being “relapsed.” Symptoms of rheumatoid arthritis, without limitation, include any of the symptoms listed in the 2000 revised American Rheumatoid Association criteria for the classification of rheumatoid arthritis, such as, for example, joint swelling, joint damage, bone deformity, bone erosion, pain, inflammation, and systemic effects associated with more advance forms of rheumatoid arthritis, including hypoalbuminemia and anemia of chronic disease. The patient is considered to be relapsed, if one or more of these symptoms return, following initial successful treatment.

### II. Treatment Methods

The present invention concerns a new mode of administration of CD20 antagonists to human subjects to treat an autoimmune disease. Thus, in one embodiment, the invention concerns a new treatment protocol for the administration of CD20 antibodies to treat an autoimmune disease, such as a chronic CD20-associated autoimmune disease, e.g. rheumatoid arthritis (RA) or lupus.

If the CD20 antibody is Rituxan® the current protocol includes two-1000 mg i.v. infusions separated by 2 weeks. In contrast, the treatment protocol of the present invention, as discussed above, comprises, consists essentially of or consist of the administration of a full effective dose at the outset in the form of an i.v injection.

According to the present invention, an effective amount of the CD20 antagonist, such as a CD20 antibody, is administered in the form of a first single intravenous (i.v.) infusion of the full dosage at the beginning of the treatment, optionally followed by a second i.v. infusion of a full effective dose (usually but, not necessarily, essentially the same dosage as the first one) about 3 to 7 months, typically about 4 to 6 months following the first administration. If the autoimmune disease treated is active RA, the second i.v. administration takes place usually 6 month after the initial treatment. In the case of lupus, the second i.v. administration is usually scheduled 4 months or 16 weeks after the initial treatment, however, the timing of the second administration might vary depending on the nature and stage of the autoimmune disease to be treated, and the patient's response to the initial treatment which might vary from individual to individual. While the second effective dose is typically the same as the first one, it might be greater or smaller, depending on the patient's response to the first treatment, and the resultant change in the patient's condition.

CD20 antibodies include: “C2B8,” which is now called “rituximab” (“RITUXAN®”) (U.S. Pat. No. 5,736,137); the yttrium-[90]-labelled 2B8 murine antibody designated “Y2B8” or “Ibritumomab Tiuxetan” (ZEVALIN®) commercially available from IDEC Pharmaceuticals, Inc. (U.S. Pat. No. 5,736,137; 2B8 deposited with ATCC under accession no. HB11388 on Jun. 22, 1993); murine IgG2a “B1,” also called “Tositumomab,” optionally labelled with ¹³¹I to generate the “131I-B1” or “iodine 1131 tositumomab” antibody (BEXXAR™) commercially available from Corixa (see, also, U.S. Pat. No. 5,595,721); murine monoclonal antibody “IF5” (Press et al. Blood 69(2):584-591 (1987) and variants thereof including “framework patched” or humanized IF5 (WO 2003/002607, Leung, S.; ATCC deposit HB-96450); murine 2H7 and chimeric 2H7 antibody (U.S. Pat. No. 5,677,180); a humanized 2H7 (WO 2004/056312 Lowman et al.) and as set forth below); HUMAX-CD20™ fully human antibody (Genmab, Denmark; see, for example, Glennie and van de Winkel, Drug Discovery Today 8: 503-510 (2003) and Cragg et al., Blood 101: 1045-1052 (2003)); the human monoclonal antibodies set forth in WO 2004/035607 (Teeling et al.); the antibodies having complex N-glycoside-linked sugar chains bound to the Fc region described in US 2004/0093621 (Shiara et al.); CD20 binding molecules such as the AME series of antibodies, e.g., AME-133™ antibodies as set forth in WO 2004/103404 (Watkins et al., Applied Molecular Evolution); A20 antibody or variants thereof such as chimeric or humanized A20 antibody (cA20, hA20, respectively) (US 2003/0219433, Immunomedics); and monoclonal antibodies L27, G28-2, 93-1B3, B-C1 or NU-B2 available from the International Leukocyte Typing Workshop (Valentine et al., In: Leukocyte Typing III (McMichael, Ed., p. 440, Oxford University Press (1987)). The preferred CD20 antibodies herein are humanized, chimeric, or human CD20 antibodies, more preferably, a humanized 2H7 antibody, rituximab, chimeric or humanized A20 antibody (Immunomedics), and HUMAX-CD20™ human CD20 antibody (Genmab).

A humanized antibody that binds human CD20 and preferably other primate CD20 as well, will comprise a heavy (H) chain having at least one, preferably two or all of the H chain CDRs of a non-human species anti-human CD20 antibody (donor antibody), and substantially all of the framework residues of a human consensus antibody as the recipient antibody. The donor antibody can be from various non-human species including mouse, rat, guinea pig, goat, rabbit, horse, primate but most frequently will be a murine antibody. “Substantially all” in this context is meant that the recipient FR regions in the humanized antibody may include one or more amino acid substitutions not originally present in the human consensus FR sequence. These FR changes may comprise residues not found in the recipient or the donor antibody.

In one embodiment, the donor antibody is the murine 2H7 antibody, the V region including the CDR and FR sequences of each of the H and L chains of which are shown in FIGS. 1A and 1B. In a specific embodiment, the residues for the human Fab framework correspond to the consensus sequence of human Vκ subgroup I and of V_{H} subgroup III, these consensus sequences are shown in FIG. 1A and FIG. 1B, respectively. The humanized 2H7 antibody of the invention will have at least one of the CDRs in the H chain of the murine donor antibody. In one embodiment, the humanized 2H7 antibody that binds human CD20 comprises the CDRs of both the H and L chains of the donor antibody.

In a full length antibody, the humanized CD20 binding antibody of the invention will comprise a humanized V domain joined to a C domain of a human immunoglobulin. In a preferred embodiment, the H chain C region is from human IgG, preferably IgG1 or IgG3. The L chain C domain is preferably from human K, chain.

Residue numbering is according to Kabat et al., Sequences of Immunological Interest. 5th Ed. Public Health Service, National Institutes of Health, Bethesda, Md. (1991), with insertions shown as a, b, c, d, and e, and gaps shown as dashes in the sequence figures. In the CD20 binding antibodies that comprise Fc region, the C-terminal lysine (residue 447 according to the EU numbering system) of the Fc region may be removed, for example, during purification of the Ab or by recombinant engineering the nucleic acid encoding the antibody polypeptide. Accordingly, a CD20 binding antibody composition useful in this invention can comprise antibody with K447, with all K447 removed, or a mixture of antibody with and without the K447 residue.

Construction and production of the humanized 2H7 antibodies that bind CD20 have been described in WO 04/056312 and US 2006/0034835, the entire disclosure of which is incorporated herein by reference.

In specific embodiments, the humanized 2H7 antibody is an antibody listed in Table 1.

TABLE 1
Humanized anti-CD20 Antibody and Variants Thereof

| 2H7 | V_{L} | V_{H} | Full L chain | Full H chain |
|---|---|---|---|---|
| variant | SEQ ID NO. | SEQ ID NO. | SEQ ID NO. | SEQ ID NO. |
| A | 1 | 2 | 6 | 7 |
| B | 1 | 2 | 6 | 8 |
| C | 3 | 4 | 9 | 10 |
| D | 3 | 4 | 9 | 11 |
| F | 3 | 4 | 9 | 12 |
| G | 3 | 4 | 9 | 13 |
| H | 3 | 5 | 9 | 14 |
| I | 1 | 2 | 6 | 15 |

Each of the antibody variants A, B and I of Table 1 comprises the light chain variable sequence (V_{L}):

(SEQ ID NO: 1)

DIQMTQSPSSLSASVGDRVTITCRASSSVSYMHWYQQKPGKAPKPLIYAP
SNLASGVPSRFSGSGSGTDFTLTISSLQPEDFATYYCQQWSFNPPTFGQG
TKVEIKR;
and

the heavy chain variable sequence (V_{H}):

(SEQ ID NO:2)

EVQLVESGGGLVQPGGSLRLSCAASGYTFTSYNMHWVRQAPGKGLEWVGA
IYPGNGDTSYNQKFKGRFTISVDKSKNTLYLQMNSLRAEDTAVYYCARVV
YYSNSYWYFDVWGQGTLVTVSS.

Each of the antibody variants C, D, F and G of Table 1 comprises the light chain variable sequence (V_{L}):

(SEQ ID NO: 3)

DIQMTQSPSSLSASVGDRVTITCRASSSVSYLHWYQQKPGKAPKPLIYAP
SNLASGVPSRFSGSGSGTDFTLTISSLQPEDFATYYCQQWAFNPPTFGQG
TKVEIKR,
and

the heavy chain variable sequence (V_{H}):

(SEQ ID NO:4)

EVQLVESGGGLVQPGGSLRLSCAASGYTFTSYNMHWVRQAPGKGLEWVGA
IYPGNGATSYNQKFKGRFTISVDKSKNTLYLQMNSLRAEDTAVYYCARVV
YYSASYWYFDVWGQGTLVTVSS.

The antibody variant H of Table 1 comprises the light chain variable sequence (V_{L}) of SEQ ID NO:3 (above) and the heavy chain variable sequence (V_{H}):

(SEQ ID NO:5)

EVQLVESGGGLVQPGGSLRLSCAASGYTFTSYNMHWVRQAPGKGLEWVGA
IYPGNGATSYNQKFKGRFTISVDKSKNTLYLQMNSLRAEDTAVYYCARVV
YYSYRYWYFDVWGQGTLVTVSS

Each of the antibody variants A, B and I of Table 1 comprises the full length light chain sequence:

(SEQ ID NO:6)

DIQMTQSPSSLSASVGDRVTITCRASSSVSYMHWYQQKPGKAPKPLIYAP
SNLASGVPSRFSGSGSGTDFTLTISSLQPEDFATYYCQQWSFNPPTFGQG
TKVEIKRTVAAPSVFIFPPSDEQLKSGTASVVCLLNNFYPREAKVQWKVD
NALQSGNSQESVTEQDSKDSTYSLSSTLTLSKADYEKHKVYACEVTHQGL
SSPVTKSFNRGEC.

Variant A of Table 1 comprises the full length heavy chain sequence:

(SEQ ID NO:7)

EVQLVESGGGLVQPGGSLRLSCAASGYTFTSYNMHWVRQAPGKGLEWVGA
IYPGNGDTSYNQKFKGRFTISVDKSKNTLYLQMNSLRAEDTAVYYCARVV
YYSNSYWYFDVWGQGTLVTVSSASTKGPSVFPLAPSSKSTSGGTAALGCL
VKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPP
KPKDTLMISRTPEVTCVVVDVSHEDPEVKFNWYVDGVEVHNAKTKPREEQ
YNSTYRVVSVLTVLHQDWLNGKEYKCKVSNKALPAPIEKTISKAKGQPRE
PQVYTLPPSREEMTKNQVSLTCLVKGFYPSDIAVEWESNGQPENNYKTTP
PVLDSDGSFFLYSKLTVDKSRWQQGNVFSCSVMHEALHNHYTQKSLSLSP
GK.

Variant I of Table 1 comprises the full length heavy chain sequence:

(SEQ ID NO:15)

EVQLVESGGGLVQPGGSLRLSCAASGYTFTSYNMHWVRQAPGKGLEWVGA
IYPGNGDTSYNQKFKGRFTISVDKSKNTLYLQMNSLRAEDTAVYYCARVV
YYSNSYWYFDVWGQGTLVTVSSASTKGPSVFPLAPSSKSTSGGTAALGCL
VKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPP
KPKDTLMISRTPEVTCVVVDVSHEDPEVKFNWYVDGVEVHNAKTKPREEQ
YNATYRVVSVLTVLHQDWLNGKEYKCKVSNAALPAPIAATISKAKGQPRE
PQVYTLPPSREEMTKNQVSLTCLVKGFYPSDIAVEWESNGQPENNYKTTP
PVLDSDGSFFLYSKLTVDKSRWQQGNVFSCSVMHEALHNHYTQKSLSLSP
GK.

Variant B of Table 1 comprises the full length heavy chain sequence:

(SEQ ID NO:8)

EVQLVESGGGLVQPGGSLRLSCAASGYTFTSYNMHWVRQAPGKGLEWVGA
IYPGNGDTSYNQKFKGRFTISVDKSKNTLYLQMNSLRAEDTAVYYCARVV
YYSNSYWYFDVWGQGTLVTVSSASTKGPSVIFPLAPSSKSTSGGTAALGC
LVKDYFPEPVTVSWNSGALTSGVHTFPAVLQSSGLYSLSSVVTVPSSSLG
TQTYICNVNHKPSNTKVDKKVEPKSCDKTHTCPPCPAPELLGGPSVFLFP
PKPKDTLMISRTPEVTCVVVDVSHEDPEVKFNWYVDGVEVHNAKTKIPRE
EQYNATYRVVSVLTVLHQDWLNGKEYKCKVSNKALPAPIAATISKAKGQP
REPQVYTLPPSREEMTKNQVSLTCLVKGFYPSDIIAVEWESNGQPENNYK
TTPPVLDSDGSFFLYSKLTVDKSRWQQGNVFSCSVMHEALHNHYTQKSLS
LSPGK.

Each of the antibody variants C. D, F, G and H of Table 1 comprises the full length light chain sequence:

(SEQ ID NO:9)

DIQMTQSPSSLSASVGDRVTITCRASSSVSYLHWYQQKPGKAPKPLIYAP
SNLASGVPSRFSGSGSGTDFTLTISSLQPEDFATYYCQQWAFNPPTFGQG
TKVEIKRTVAAPSVFIFPPSDEQLKSGTASVVCLLNNFYPREAKVQWKVD
NALQSGNSQESVTEQDSKDSTYSLSSTLTLSKADYEKHKVYACEVTHQGL
SSPVTKSFNRGEC.

Variant C of Table 1 comprises the full length heavy chain sequence:

(SEQ ID NO:10)

EVQLVESGGGLVQPGGSLRLSCAASGYTFTSYNMHWVRQAPGKGLEWVGA
IYPGNGATSYNQKFKGRFTISVDKSKNTLYLQMNSLRAEDTAVYYCARVV
YYSASYWYFDVWGQGTLVTVSSASTKGPSVFPLAPSSKSTSGGTAALGCL
VKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPP
KPKDTLMISRTPEVTCVVVDVSHEDPEVKFNWYVDGVEVHNAKTKPREEQ
YNATYRVVSVLTVLHQDWLNGKEYKCKVSNKALPAPIAATISKAKGQPRE
PQVYTLPPSREEMTKNQVSLTCLVKGFYPSDIAVEWESNGQPENNYKTTP
PVLDSDGSFFLYSKLTVDKSRWQQGNVFSCSVMHEALHNHYTQKSLSLSP
GK.

Variant D of Table 1 comprises the full length heavy chain sequence:

(SEQ ID NO:11)

EVQLVESGGGLVQPGGSLRLSCAASGYTFTSYNMHWVRQAPGKGLEWVGA
IYPGNGATSYNQKFKGRFTISVDKSKNTLYLQMNSLRAEDTAVYYCARVV
YYSASYWYFDVWGQGTLVTVSSASTKGPSVFPLAPSSKSTSGGTAALGCL
VKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPP
KPKDTLMISRTPEVTCVVVDVSHEDPEVKFNWYVDGVEVHNAKTKPREEQ
YNATYRVVSVLTVLHQDWLNGKEYKCAVSNKALPAPIEATISKAKGQPRE
PQVYTLPPSREEMTKNQVSLTCLVKGFYPSDIAVEWESNGQPENNYKTFI
TPPVLDSDGSFFLYSKLTVDKSRWQQGNVFSCSVMHEALHNHYTQKSLSL
SPGK.

Variant F of Table 1 comprises the full length heavy chain sequence:

(SEQ ID NO:12)

EVQLVESGGGLVQPGGSLRLSCAASGYTFTSYNMHWVRQAPGKGLEWVGA
IYPGNGATSYNQKFKGRFTISVDKSKNTLYLQMNSLRAEDTAVYYCARVV
YYSASYWYFDVWGQGTLVTVSSASTKGPSVFPLAPSSKSTSGGTAALGCL
VKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPP
KPKDTLMISRTPEVTCVVVDVSHEDPEVKFNWYVDGVEVHNAKTKPREEQ
YNATYRVVSVLTVLHQDWLNGKEYKCKVSNAALPAPIAATISKAKGQPRE
PQVYTLPPSREEMTKNQVSLTCLVKGFYPSDIAVEWESNGQPENNYKTTP
PVLDSDGSFFLYSKLTVDKSRWQQGNVFSCSVMHEALHNHYTQKSLSLSP
GK.

Variant G of Table 1 comprises the full length heavy chain sequence:

(SEQ ID NO:13)

EVQLVESGGGLVQPGGSLRLSCAASGYTFTSYNMHWVRQAPGKGLEWVGA
IYPGNGATSYNQKFKGRFTISVDKSKNTLYLQMNSLRAEDTAVYYCARVV
YYSASYWYFDVWGQGTLVTVSSASTKGPSVFPLAPSSKSTSGGTAALGCL
VKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPP
KPKDTLMISRTPEVTCVVVDVSHEDPEVKFNWYVDGVEVHNAKTKPREEQ
YNATYRVVSVLTVLHQDWLNGKEYKCKVSNAALPAPIAATISKAKGQPRE
PQVYTLPPSREEMTKNQVSLTCLVKGFYPSDIAVEWESNGQPENNYKTTP
PVLDSDGSFFLYSKLTVDKSRWQQGNVFSCSVMHEALHWHYTQKSLSLSP
GK.

Variant H of Table 1 comprises the full length heavy chain sequence:

(SEQ ID NO:14)

EVQLVESGGGLVQPGGSLRLSCAASGYTFTSYNMHWVRQAPGKGLEWVGA
IYPGNGATSYNQKFKGRFTISVDKSKNTLYLQMNSLRAEDTAVYYCARVV
YYSYRYWYFDVWGQGTLVTVSSASTKGPSVFPLAPSSKSTSGGTAALGCL
VKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPP
KPKDTLMISRTPEVTCVVVDVSHEDPEVKFNWYVDGVEVHNAKTKPREEQ
YNATYRVVSVLTVLHQDWLNGKEYKCKVSNAALPAPIAATISKAKCQPRE
PQVYTLPPSREEMTKNQVSLTCLVKGFYPSDIAVEWESNGQPENNYKTFI
TPPVLDSDGSFFLYSKLTVDKSRWQQGNVFSCSVMHEALHNHYTQKSLSL
SPGK.

In certain embodiments, the humanized 2H7 antibody of the invention further comprises amino acid alterations in the IgG Fc and exhibits increased binding affinity for human FcRn over an antibody having wild-type IgG Fc, by at least 60 fold, at least 70 fold, at least 80 fold, more preferably at least 100 fold, preferably at least 125 fold, even more preferably at least 150 fold to about 170 fold.

The N-glycosylation site in IgG is at Asn297 in the C_{H}2 domain. Humanized 2H7 antibody compositions of the present invention include compositions of any of the preceding humanized 2H7 antibodies having a Fc region, wherein about 80-100% (and preferably about 90-99%) of the antibody in the composition comprises a mature core carbohydrate structure which lacks fucose, attached to the Fc region of the glycoprotein. Such compositions were demonstrated herein to exhibit a surprising improvement in binding to Fc(RIIIA(F158), which is not as effective as Fc(RIIIA (VI58) in interacting with human IgG. Fc(RIIIA (F158) is more common than Fc(RIIIA (VI58) in normal, healthy African Americans and Caucasians. See Lehinbecher et al., Blood 94:4220 (1999). Historically, antibodies produced in Chinese Hamster Ovary Cells (CHO), one of the most commonly used industrial hosts, contain about 2 to 6% in the population that are nonfucosylated. YB2/0 and Lec13, however, can produce antibodies with 78 to 98% nonfucosylated species. Shinkawa et al., J Bio. Chem. 278 (5), 3466-347 (2003), reported that antibodies produced in YB2/0 and Lec13 cells, which have less FUT8 activity, show significantly increased ADCC activity in vitro. The production of antibodies with reduced fucose content are also described in e.g., Li et al., (GlycoFi) “Optimization of humanized IgGs in glycoengineered Pichia pastoris” in Nature Biology online publication 22 Jan. 2006; Niwa R. et al., Cancer Res. 64(6):2127-2133 (2004); US 2003/0157108 (Presta); U.S. Pat. No. 6,602,684 and US 2003/0175884 (Glycart Biotechnology); US 2004/0093621, US 2004/0110704, US 2004/0132140 (all of Kyowa Hakko Kogyo).

CD20 binding antibodies encompass bispecific CD20 binding antibodies wherein one arm of the antibody has a heavy (H) and a light (L) chain of a CD20 binding antibody such as a H and L chain of the humanized 2H7 antibody of the invention, and the other arm has V region binding specificity for a second antigen. In specific embodiments, the second antigen is selected from the group consisting of CD3, CD64, CD32A, CD16, NKG2D or other NK activating ligands.

The Genentech and Biogen Idec clinical investigations have evaluated the therapeutic effectiveness of treatment of autoimmune diseases using doses of anti-CD20 (humanized 2H7 variant and Rituximab) ranging from as low as 10 mg up to a dose of 1 g (see under Background section for Rituximab studies; and WO 04/056312, Example 16). In general, the antibodies were administered in these clinical investigations in two doses, spaced about two weeks apart. Examples of regimens studied in the clinical investigations include, for humanized CD20 antibody 2H7 in rheumatoid arthritis at 2×10 mg (means 2 doses at 10 mg per dose; total dose of ˜10.1 mg/m² for a 70 kg, 67 inch tall patient), 2×50 mg (total dose of 55 mg/m² for a 70 kg, 67 in tall patient), 2×200 mg (total dose of 220 mg/m² for a 70 kg, 67 in tall patient), 2×500 mg (total dose of 550 mg/m2 for a 70 kg, 67 in tall patient) and 2×1000 mg (total dose of ˜100 mg/m2 for a 70 kg, 67 in tall patient); and for Rituxan, 2×500 mg (total dose of ˜550 mg/m2 for a 70 kg, 67 in tall patient), 2×1000 mg (total dose of 1100 mg/m2 for a 70 kg, 67 in tall patient).

The therapeutic effectiveness of treatment of autoimmune diseases has been evaluated using doses of CD20 antibody ranging from as low as 1 mg up to a dose of 2 g. Since, as shown in the Example, the single infusion treatment showed similar results for all dosages tested (400 mg, 1000 mg, 1500 mg and 2000 mg), the effective dose might vary within a wide range and typically is between about 0.1 mg and about 2000 mg, or between about 1 mg and about 2000 mg, or between about 10 mg and 2000 mg, or between about 400 mg and 2000 mg of the CD20 antibody. The effective dose varies depending on the CD20 antibody used, the disease treated, including the severity of the disease, the patient's sex, weight, age and overall physical condition, and other factors, usually considered by practicing physicians. Determination of the effective dose for each particular situation is well within the skill of an ordinary physician. Thus, in various embodiments, the CD20 antibody can be administered at dosages of 0.1, 0.5, 1, 5, 10, 15, 20 25, 30, 40, 50, 75, 100, 125, 150, 200, 250, 300, 350, 400, 500, 1000, 1500, or 2000 mg. The parameters for assessing efficacy or success of treatment of an autoimmune or autoimmune related disease will be known to the physician of skill in the appropriate disease. Generally, the physician of skill will look for reduction in the signs and symptoms of the specific disease.

For treatment of an autoimmune disease, it may be desirable to modulate the extent of B cell depletion depending on the disease and/or the severity of the condition in the individual patient, by adjusting the dosage of CD20 binding antibody. Thus, B cell depletion can but does not have to be complete. Or, total B cell depletion may be desired in initial treatment but in subsequent treatments, the dosage may be adjusted to achieve only partial depletion. In one embodiment, the B cell depletion is at least 20%, i.e., 80% or less of CD20 positive B cells remain as compared to the baseline level before treatment. In other embodiments, B cell depletion is 25%, 30%, 40%, 50%, 60%, 70% or greater. Preferably, the B cell depletion is sufficient to halt progression of the disease, more preferably to alleviate the signs and symptoms of the particular disease under treatment, even more preferably to cure the disease. Lower CD20 dosages e.g., at 20 mg, 10 mg or lower, can be used if partial or short term B cell depletion is the objective, or if the disease is not chronic or less severe. In another embodiment, treatment at the low dosages of the present invention is useful in maintenance therapy, following successful treatment with an initial higher dosage, administered in accordance with the present invention. In a particular embodiment, the first administration of a complete effective dose as a single i.v. infusion is followed by the i.v. infusion is the same dose 4 to 6 months after the first administration, which may be followed by the administration of lower maintenance dosages, typically in 4 to 6 months intervals.

Patients having an autoimmune disease for whom one or more current standard of care therapies were ineffective, poorly tolerated, or contraindicated can be treated using the dosing regimens of the present invention. For example, the invention contemplates the present treatment methods for RA patients who have had an inadequate response to tumor necrosis factor (TNF) inhibitor therapies or to disease-modifying anti-rheumatic drugs (DMARD) therapy.

In one embodiment, the present dosages and dosing regimen are used in treating rheumatoid arthritis (RA), such as active RA, e.g. moderate to severe active RA.

RA is a debilitating autoimmune disease that affects more than two million Americans and hinders the daily activities of sufferers. RA occurs when the body's own immune system inappropriately attacks joint tissue and causes chronic inflammation that destroys healthy tissue and damage within the joints. Symptoms include inflammation of the joints, swelling, stiffness, and pain. Additionally, since RA is a systemic disease, it can have effects in other tissues such as the lungs, eyes and bone marrow.

At present, there is no known cure for RA. Current treatments include a variety of steroidal and non-steroidal anti-inflammatory drugs, immunosuppressive agents, disease-modifying anti-rheumatic drugs (DMARDs), and biologics. However, many patients continue to have an inadequate response to such treatments.

The CD20 antibodies can be used as first-line therapy in patients with early RA (i.e., methotrexate (MTX) naive) and as monotherapy, or in combination with, e.g., MTX or cyclophosphamide. Or, the antibodies can be used in treatment as second-line therapy for patients who were DMARD and/or MTX refractory, and as monotherapy or in combination with other drug therapies, e.g., MTX. The humanized CD20 binding antibodies are useful to prevent and control joint damage, delay structural damage, decrease pain associated with inflammation in RA, and generally reduce the signs and symptoms in active RA, such as moderate to severe RA. The RA patient can be treated with the humanized CD20 antibody prior to, after or together with treatment with other drugs used in treating RA (see combination therapy below). In one embodiment, patients who had previously failed disease-modifying antirheumatic drugs and/or had an inadequate response to methotrexate alone are treated with a humanized CD20 binding antibody herein. In one embodiment of this treatment, the patients initially receive a humanized CD20 binding antibody alone, following the dosing regimen of the present invention, followed by the administration of the CD20 antibody plus cyclophosphamide, or CD20 antibody plus methotrexate.

One method of evaluating treatment efficacy in RA is based on American College of Rheumatology (ACR) criteria, which measures the percentage of improvement in tender and swollen joints, among other things. The RA patient can be scored at for example, ACR 20 (20 percent improvement) compared with no antibody treatment (e.g, baseline before treatment) or treatment with placebo. Other ways of evaluating the efficacy of antibody treatment include X-ray scoring such as the Sharp X-ray score used to score structural damage such as bone erosion and joint space narrowing. Patients can also be evaluated for the prevention of or improvement in disability based on Health Assessment Questionnaire [HAQ] score, AIMS score, SF-36 at time periods during or after treatment. The ACR 20 criteria may include 20% improvement in both tender (painful) joint count and swollen joint count plus a 20% improvement in at least 3 of 5 additional measures:
1. patient's pain assessment by visual analog scale (VAS),
2. patient's global assessment of disease activity (VAS),
3. physician's global assessment of disease activity (VAS),
4. patient's self-assessed disability measured by the Health Assessment Questionnaire, and
5. acute phase reactants, CRP or ESR.

The ACR 50 and 70 are defined analogously. Preferably, the patient is administered an amount of a CD20 antibody of the invention effective to achieve at least a score of ACR 20, preferably at least ACR 30, more preferably at least ACR50, even more preferably at least ACR70, most preferably at least ACR 75 and higher.

Psoriatic arthritis has unique and distinct radiographic features. For psoriatic arthritis, joint erosion and joint space narrowing can be evaluated by the Sharp score as well. The humanized CD20 binding antibodies of the invention can be used to prevent the joint damage as well as reduce disease signs and symptoms of the disorder.

Yet another aspect of the invention is a method of treating Lupus or SLE by administering to the patient suffering from SLE, a therapeutically effective amount of a humanized CD20 antibody of the invention. SLE patients include patients with extra-renal manifestations as well as with lupus nephritis. SLEDAI scores provide a numerical quantitation of disease activity. The SLEDAI is a weighted index of 24 clinical and laboratory parameters known to correlate with disease activity, with a numerical range of 0-103. See Bryan Gescuk & John Davis, “Novel therapeutic agent for systemic lupus erythematosus” in Current Opinion in Rheumatology 2002, 14:515-521. Antibodies to double-stranded DNA are believed to cause renal flares and other manifestations of lupus. Patients undergoing antibody treatment can be monitored for time to renal flare, which is defined as a significant, reproducible increase in serum creatinine, urine protein or blood in the urine. Alternatively or in addition, patients can be monitored for levels of antinuclear antibodies and antibodies to double-stranded DNA. Treatments for SLE include high-dose corticosteroids and/or cyclophosphamide (HDCC).

With regard to vasculitis, approximately 75% of the patients with systemic vasculitides have anti-neutrophil cytoplasmic antibody and cluster into one of three conditions affecting small/medium sized vessels: Wegeners granulomatosus (WG), microscopic polyangiitis (MPA) and Churg Strauss syndrome (CSS), collectively known as ANCA associated vasculitis (AAV).

Spondyloarthropathies are a group of disorders of the joints, including ankylosing spondylitis, psoriatic arthritis and Crohn's disease. Treatment success can be determined by validated patient and physician global assessment measuring tools.

Various medications are used to treat psoriasis; treatment differs directly in relation to disease severity. Patients with a more mild form of psoriasis typically utilize topical treatments, such as topical steroids, anthralin, calcipotriene, clobetasol, and tazarotene, to manage the disease while patients with moderate and severe psoriasis are more likely to employ systemic (methotrexate, retinoids, cyclosporine, PUVA and UVB) therapies. Tars are also used. These therapies have a combination of safety concerns, time consuming regimens, or inconvenient processes of treatment. Furthermore, some require expensive equipment and dedicated space in the office setting. Systemic medications can produce serious side effects, including hypertension, hyperlipidemia, bone marrow suppression, liver disease, kidney disease and gastrointestinal upset. Also, the use of phototherapy can increase the incidence of skin cancers. In addition to the inconvenience and discomfort associated with the use of topical therapies, phototherapy and systemic treatments require cycling patients on and off therapy and monitoring lifetime exposure due to their side effects.

Treatment efficacy for psoriasis is assessed by monitoring changes in clinical signs and symptoms of the disease including Physician's Global Assessment (PGA) changes and Psoriasis Area and Severity Index (PASI) scores, Psoriasis Symptom Assessment (PSA), compared with the baseline condition. The patient can be measured periodically throughout treatment on the Visual analog scale used to indicate the degree of itching experienced at specific time points.

Patients may experience an infusion reaction or infusion-related symptoms with their first infusion of a therapeutic antibody. These symptoms vary in severity and generally are reversible with medical intervention. These symptoms include but are not limited to, flu-like fever, chills/rigors, nausea, urticaria, headache, bronchospasm, angioedema. It would be desirable for the disease treatment methods of the present invention to minimize infusion reactions. To alleviate or minimize such adverse events, the patient may receive an initial conditioning or tolerizing dose(s) of the antibody followed by a therapeutically effective dose. The conditioning dose(s) will be lower than the therapeutically effective dose to condition the patient to tolerate higher dosages.

In treating an autoimmune disease, such as the autoimmune diseases or autoimmune related conditions described above, the patient can be treated with one or more CD20 binding antibodies in conjunction with a second therapeutic agent, such as an immunosuppressive agent, such as in a multi drug regimen. The CD20 binding antibody can be administered concurrently, sequentially or alternating with the immunosuppressive agent or upon non-responsiveness with other therapy. The immunosuppressive agent can be administered at the same or lesser dosages than as set forth in the art. The preferred adjunct immunosuppressive agent will depend on many factors, including the type of disorder being treated as well as the patient's history.

“Immunosuppressive agent” as used herein for adjunct therapy refers to substances that act to suppress or mask the immune system of a patient. Such agents would include substances that suppress cytokine production, down regulate or suppress self-antigen expression, or mask the MHC antigens. Examples of such agents include steroids such as glucocorticosteroids, e.g., prednisone, methylprednisolone, and dexamethasone; 2-amino-6-aryl-5-substituted pyrimidines (see U.S. Pat. No. 4,665,077), azathioprine (or cyclophosphamide, if there is an adverse reaction to azathioprine); bromocryptine; glutaraldehyde (which masks the MHC antigens, as described in U.S. Pat. No. 4,120,649); anti-idiotypic antibodies for MHC antigens and MHC fragments; cyclosporin A; cytokine or cytokine receptor antagonists including anti-interferon-α, -β, or -γ antibodies; anti-tumor necrosis factor-α antibodies; anti-tumor necrosis factor-β antibodies; anti-interleukin-2 antibodies and anti-IL-2 receptor antibodies; anti-L3T4 antibodies; heterologous anti-lymphocyte globulin; pan-T antibodies, preferably anti-CD3 or anti-CD4/CD4a antibodies; soluble peptide containing a LFA-3 binding domain (WO 90/08187 published Jul. 26, 1990); streptokinase; TGF-β; streptodomase; RNA or DNA from the host; FK506; RS-61443; deoxyspergualin; rapamycin; T-cell receptor (U.S. Pat. No. 5,114,721); T-cell receptor fragments (Offner et al., Science 251:430-432 (1991); WO 90/11294; and WO 91/01133); and T cell receptor antibodies (EP 340,109) such as T10B9.

For the treatment of rheumatoid arthritis, the patient can be treated with a CD20 binding antibody (such as rituximab or ocrelizumab or variant thereof) in conjunction with any one or more of the following drugs: DMARDS (disease-modifying anti-rheumatic drugs (e.g., methotrexate), NSAI or NSAID (non-steroidal anti-inflammatory drugs), immunosuppressants (e.g., azathioprine; mycophenolate mofetil (CellCept®; Roche)), analgesics, glucocorticosteroids, cyclophosphamide, HUMIRA™ (adalimumab; Abbott Laboratories), ARAVA® (leflunomide), REMICADE® (infliximab; Centocor Inc., of Malvern, Pa.), ENBREL (etanercept; Immunex, Wash.), ACTEMRA (tocilizumab; Roche, Switzerland), COX-2 inhibitors. DMARDs commonly used in RA are hydroxycloroquine, sulfasalazine, methotrexate, leflunomide, etanercept, infliximab, azathioprine, D-penicillamine, Gold (oral), Gold (intramuscular), minocycline, cyclosporine, Staphylococcal protein A immunoadsorption. Adalimumab is a human monoclonal antibody that binds to TNFV. Infliximab is a chimeric monoclonal antibody that binds to TNFV. Etanercept is an “immunoadhesin” fusion protein consisting of the extracellular ligand binding portion of the human 75 kD (p75) tumor necrosis factor receptor (TNFR) linked to the Fc portion of a human IgG1. Actemra (tocilizumab) is a humanized anti-human interleukin-6 (IL-6) receptor. For conventional treatment of RA, see, e.g., “Guidelines for the management of rheumatoid arthritis” Arthritis & Rheumatism 46(2): 328-346 (February, 2002). In a specific embodiment, the RA patient is treated with a CD20 antibody of the invention in conjunction with methotrexate (MTX). An exemplary dosage of MTX is about 7.5-25 mg/kg/wk. MTX can be administered orally and subcutaneously.

For the treatment of ankylosing spondylitis, psoriatic arthritis and Crohn's disease, the patient can be treated with a CD20 binding antibody of the invention in conjunction with, for example, Remicade® (infliximab; from Centocor Inc., of Malvern, Pa.), ENBREL (etanercept; Immunex, Wash.).

Treatments for SLE include combination of the CD20 antibody with high-dose corticosteroids and/or cyclophosphamide (HDCC). Patients suffering from SLE, AAV and NMO can be treated with a CD20 binding antibody of the invention in combination with any of the following: corticosteroids, NSAIDs, analgesics, COX-2 inhibitors, glucocorticosteriods, conventional DMARDS (e.g. methotexate, sulphasalazine, hydroxychloroquine, leflunomide), biologic DMARDs such as anti-Blys (e.g., belimumab), anti-IL6R e.g., tocilizumab; CTLA4-Ig (abatacept), (anti-CD22 e.g., epratuzumab), immunosuppressants (e.g., azathioprine; mycophenolate mofetil (CellCept®; Roche)), and cytotoxic agents (e.g., cyclophosphamide).

For the treatment of psoriasis, patients can be administered a CD20 binding antibody in conjunction with topical treatments, such as topical steroids, anthralin, calcipotriene, clobetasol, and tazarotene, or with methotrexate, retinoids, cyclosporine, PUVA and UVB therapies. In one embodiment, the psoriasis patient is treated with a CD20 binding antibody sequentially or concurrently with cyclosporine.

To minimize toxicity, the traditional systemic therapies can be administered in rotational, sequential, combinatorial, or intermittent treatment regimens, or lower dosage combination regimens with the CD20 binding antibody compositions at the present dosages.

Therapeutic formulations of the CD20-binding antibodies used in accordance with the present invention are prepared for storage by mixing an antibody having the desired degree of purity with optional pharmaceutically acceptable carriers, excipients or stabilizers (Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as olyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG).

### III. Preparation of CD20 Antibodies

The methods and articles of manufacture of the present invention use, or incorporate, an antibody that binds to a B-cell surface marker, especially one that binds to CD20. Accordingly, methods for generating such antibodies will be described here.

CD20 antigen to be used for production of, or screening for, antibody(ies) may be, e.g., a soluble form of CD20 or a portion thereof, containing the desired epitope. Alternatively, or additionally, cells expressing CD20 at their cell surface can be used to generate, or screen for, antibody(ies). Other forms of CD20 useful for generating antibodies will be apparent to those skilled in the art.

A description follows as to exemplary techniques for the production of the antibodies used in accordance with the present invention.

(i) Polyclonal Antibodies

Polyclonal antibodies are preferably raised in animals by multiple subcutaneous (s.c.) or intraperitoneal (i.p.) injections of the relevant antigen and an adjuvant. It may be useful to conjugate the relevant antigen to a protein that is immunogenic in the species to be immunized, e.g., keyhole limpet hemocyanin, serum albumin, bovine thyroglobulin, or soybean trypsin inhibitor using a bifunctional or derivatizing agent, for example, maleimidobenzoyl sulfosuccinimide ester (conjugation through cysteine residues), N-hydroxysuccinimide (through lysine residues), glutaraldehyde, succinic anhydride, SOCl₂, or R¹N═C═NR, where R and R′ are different alkyl groups.

Animals are immunized against the antigen, immunogenic conjugates, or derivatives by combining, e.g., 100 μg or 5 μg of the protein or conjugate (for rabbits or mice, respectively) with 3 volumes of Freund's complete adjuvant and injecting the solution intradermally at multiple sites. One month later the animals are boosted with ⅕ to 1/10 the original amount of peptide or conjugate in Freund's complete adjuvant by subcutaneous injection at multiple sites. Seven to 14 days later the animals are bled and the serum is assayed for antibody titer. Animals are boosted until the titer plateaus. Preferably, the animal is boosted with the conjugate of the same antigen, but conjugated to a different protein and/or through a different cross-linking reagent. Conjugates also can be made in recombinant cell culture as protein fusions. Also, aggregating agents such as alum are suitably used to enhance the immune response.

(ii) Monoclonal Antibodies

Monoclonal antibodies are obtained from a population of substantially homogeneous antibodies, i.e., the individual antibodies comprising the population are identical and/or bind the same epitope except for possible variants that arise during production of the monoclonal antibody, such variants generally being present in minor amounts. Thus, the modifier “monoclonal” indicates the character of the antibody as not being a mixture of discrete or polyclonal antibodies.

For example, the monoclonal antibodies may be made using the hybridoma method first described by Kohler et al., Nature, 256:495 (1975), or may be made by recombinant DNA methods (U.S. Pat. No. 4,816,567).

In the hybridoma method, a mouse or other appropriate host animal, such as a hamster, is immunized as hereinabove described to elicit lymphocytes that produce or are capable of producing antibodies that will specifically bind to the protein used for immunization. Alternatively, lymphocytes may be immunized in vitro. Lymphocytes then are fused with myeloma cells using a suitable fusing agent, such as polyethylene glycol, to form a hybridoma cell (Goding, Monoclonal Antibodies: Principles and Practice, pp. 59-103 (Academic Press, 1986)).

The hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

Preferred myeloma cells are those that fuse efficiently, support stable high-level production of antibody by the selected antibody-producing cells, and are sensitive to a medium such as HAT medium. Among these, preferred myeloma cell lines are murine myeloma lines, such as those derived from MOPC-21 and MPC-11 mouse tumors available from the Salk Institute Cell Distribution Center, San Diego, Calif. USA, and SP-2 or X63-Ag8-653 cells available from the American Type Culture Collection, Rockville, Md. USA. Human myeloma and mouse-human heteromyeloma cell lines also have been described for the production of human monoclonal antibodies (Kozbor, J. Immunol., 133:3001 (1984); Brodeur et al., Monoclonal Antibody Production Techniques and Applications, pp. 51-63 (Marcel Dekker, Inc., New York, 1987)).

Culture medium in which hybridoma cells are growing is assayed for production of monoclonal antibodies directed against the antigen. Preferably, the binding specificity of monoclonal antibodies produced by hybridoma cells is determined by immunoprecipitation or by an in vitro binding assay, such as radioimmunoassay (RIA) or enzyme-linked immunoabsorbent assay (ELISA).

The binding affinity of the monoclonal antibody can, for example, be determined by the Scatchard analysis of Munson et al., Anal. Biochem., 107:220 (1980).

After hybridoma cells are identified that produce antibodies of the desired specificity, affinity, and/or activity, the clones may be subcloned by limiting dilution procedures and grown by standard methods (Goding, Monoclonal Antibodies: Principles and Practice, pp. 59-103 (Academic Press, 1986)). Suitable culture media for this purpose include, for example, D-MEM or RPMI-1640 medium. In addition, the hybridoma cells may be grown in vivo as ascites tumors in an animal.

The monoclonal antibodies secreted by the subclones are suitably separated from the culture medium, ascites fluid, or serum by conventional immunoglobulin purification procedures such as, for example, protein A-SEPHAROSE™, hydroxylapatite chromatography, gel electrophoresis, dialysis, or affinity chromatography.

DNA encoding the monoclonal antibodies is readily isolated and sequenced using conventional procedures (e.g., by using oligonucleotide probes that are capable of binding specifically to genes encoding the heavy and light chains of murine antibodies). The hybridoma cells serve as a preferred source of such DNA. Once isolated, the DNA may be placed into expression vectors, which are then transfected into host cells such as E. coli cells, simian COS cells, Chinese Hamster Ovary (CHO) cells, or myeloma cells that do not otherwise produce immunoglobulin protein, to obtain the synthesis of monoclonal antibodies in the recombinant host cells. Review articles on recombinant expression in bacteria of DNA encoding the antibody include Skerra et al., Curr. Opinion in Immunol., 5:256-262 (1993) and Pluckthun, Immunol. Revs., 130:151-188 (1992).

In a further embodiment, antibodies or antibody fragments can be isolated from antibody phage libraries generated using the techniques described in McCafferty et al., Nature, 348:552-554 (1990). Clackson et al., Nature, 352:624-628 (1991) and Marks et al., J. Mol. Biol., 222:581-597 (1991) describe the isolation of murine and human antibodies, respectively, using phage libraries. Subsequent publications describe the production of high affinity (nM range) human antibodies by chain shuffling (Marks et al., Bio/Technology, 10:779-783 (1992)), as well as combinatorial infection and in vivo recombination as a strategy for constructing very large phage libraries (Waterhouse et al., Nuc. Acids. Res., 21:2265-2266 (1993)). Thus, these techniques are viable alternatives to traditional monoclonal antibody hybridoma techniques for isolation of monoclonal antibodies.

The DNA also may be modified, for example, by substituting the coding sequence for human heavy- and light chain constant domains in place of the homologous murine sequences (U.S. Pat. No. 4,816,567; Morrison, et al., Proc. Natl Acad. Sci. USA, 81:6851 (1984)), or by covalently joining to the immunoglobulin coding sequence all or part of the coding sequence for a non-immunoglobulin polypeptide.

Typically such non-immunoglobulin polypeptides are substituted for the constant domains of an antibody, or they are substituted for the variable domains of one antigen-combining site of an antibody to create a chimeric bivalent antibody comprising one antigen-combining site having specificity for an antigen and another antigen-combining site having specificity for a different antigen.

In addition, antibodies comprising a variant Fc region with high affinity for FcγR are useful for treating diseases where an enhanced efficacy of effector cell function is desired, such as autoimmune diseases, as set forth, for example, in US 2005/0037000 and WO 2004/63351 (Macrogenics, Inc. STAVENHAGEN et al.).

(iii) Humanized Antibodies

Methods for humanizing non-human antibodies have been described in the art. Preferably, a humanized antibody has one or more amino acid residues introduced into it from a source that is non-human. These non-human amino acid residues are often referred to as “import” residues, which are typically taken from an “import” variable domain. Humanization can be essentially performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting hypervariable region sequences for the corresponding sequences of a human antibody. Accordingly, such “humanized” antibodies are chimeric antibodies (U.S. Pat. No. 4,816,567) wherein substantially less than an intact human variable domain has been substituted by the corresponding sequence from a non-human species. In practice, humanized antibodies are typically human antibodies in which some hypervariable region residues and possibly some FR residues are substituted by residues from analogous sites in rodent antibodies.

The choice of human variable domains, both light and heavy, to be used in making the humanized antibodies is very important to reduce antigenicity. According to the so-called “best-fit” method, the sequence of the variable domain of a rodent antibody is screened against the entire library of known human variable-domain sequences. The human sequence that is closest to that of the rodent is then accepted as the human framework region (FR) for the humanized antibody (Sims et al., J. Immunol., 151:2296 (1993); Chothia et al., J. Mol. Biol., 196:901 (1987)). Another method uses a particular framework region derived from the consensus sequence of all human antibodies of a particular subgroup of light or heavy chain variable regions. The same framework may be used for several different humanized antibodies (Carter et al., Proc. Natl. Acad. Sci. USA, 89:4285 (1992); Presta et al., J. Immunol., 151:2623 (1993)).

It is further important that antibodies be humanized with retention of high affinity for the antigen and other favorable biological properties. To achieve this goal, according to a preferred method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available that illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved. In general, the hypervariable region residues are directly and most substantially involved in influencing antigen binding.

(iv) Human Antibodies

As an alternative to humanization, human antibodies can be generated. For example, it is now possible to produce transgenic animals (e.g., mice) that are capable, upon immunization, of producing a full repertoire of human antibodies in the absence of endogenous immunoglobulin production. For example, it has been described that the homozygous deletion of the antibody heavy chain joining region (J_{H}) gene in chimeric and germ-line mutant mice results in complete inhibition of endogenous antibody production. Transfer of the human germ-line immunoglobulin gene array in such germ-line mutant mice will result in the production of human antibodies upon antigen challenge. See, e.g., Jakobovits et al., Proc. Natl. Acad. Sci. USA, 90:2551 (1993); Jakobovits et al., Nature, 362:255-258 (1993); Bruggermann et al., Year in Immuno., 7:33 (1993); and U.S. Pat. Nos. 5,591,669, 5,589,369 and 5,545,807.

Alternatively, phage display technology (McCafferty et al., Nature 348:552-553 (1990)) can be used to produce human antibodies and antibody fragments in vitro, from immunoglobulin variable (V) domain gene repertoires from unimmunized donors. According to this technique, antibody V domain genes are cloned in-frame into either a major or minor coat protein gene of a filamentous bacteriophage, such as M13 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimics some of the properties of the B cell. Phage display can be performed in a variety of formats; for their review see, e.g., Johnson et al., Current Opinion in Structural Biology 3:564-571 (1993). Several sources of V-gene segments can be used for phage display. Clackson et al., Nature, 352:624-628 (1991) isolated a diverse array of anti-oxazolone antibodies from a small random combinatorial library of V genes derived from the spleens of immunized mice. A repertoire of V genes from unimmunized human donors can be constructed and antibodies to a diverse array of antigens (including self-antigens) can be isolated essentially following the techniques described by Marks et al., J. Mol. Biol. 222:581-597 (1991), or Griffith et al., EMBO J. 12:725-734 (1993). See, also, U.S. Pat. Nos. 5,565,332 and 5,573,905.

Human antibodies may also be generated by in vitro activated B cells (see U.S. Pat. Nos. 5,567,610 and 5,229,275).

(v) Antibody Fragments

Various techniques have been developed for the production of antibody fragments. Traditionally, these fragments were derived via proteolytic digestion of intact antibodies (see, e.g., Morimoto et al., Journal of Biochemical and Biophysical Methods 24:107-117 (1992) and Brennan et al., Science, 229:81 (1985)). However, these fragments can now be produced directly by recombinant host cells. For example, the antibody fragments can be isolated from the antibody phage libraries discussed above. Alternatively, Fab′-SH fragments can be directly recovered from E. coli and chemically coupled to form F(ab′)₂ fragments (Carter et al., Bio/Technology 10:163-167 (1992)). According to another approach, F(ab′)₂ fragments can be isolated directly from recombinant host cell culture. Other techniques for the production of antibody fragments will be apparent to the skilled practitioner. In other embodiments, the antibody of choice is a single chain Fv fragment (scFv). See WO 93/16185; U.S. Pat. No. 5,571,894; and U.S. Pat. No. 5,587,458. The antibody fragment may also be a “linear antibody”, e.g., as described in U.S. Pat. No. 5,641,870 for example. Such linear antibody fragments may be monospecific or bispecific.

(vi) Bispecific Antibodies

Bispecific antibodies are antibodies that have binding specificities for at least two different epitopes. Exemplary bispecific antibodies may bind to two different epitopes of the CD20 antigen. Other such antibodies may bind CD20 and further bind a second B-cell surface marker. Alternatively, an anti-CD20 binding arm may be combined with an arm that binds to a triggering molecule on a leukocyte such as a T-cell receptor molecule (e.g. CD2 or CD3), or Fc receptors for IgG (FcγR), such as FcγRI (CD64), FcγRII (CD32) and FeyRIII (CD16) so as to focus cellular defense mechanisms to the B cell. Bispecific antibodies may also be used to localize certain agents to the B cell. These antibodies possess a CD20-binding arm and an arm that binds the agent (e.g. methotrexate). Bispecific antibodies can be prepared as full-length antibodies or antibody fragments (e.g. F(ab′)₂ bispecific antibodies).

Methods for making bispecific antibodies are known in the art. Traditional production of full length bispecific antibodies is based on the co-expression of two immunoglobulin heavy chain-light chain pairs, where the two chains have different specificities (Millstein et al., Nature, 305:537-539 (1983)). Because of the random assortment of immunoglobulin heavy and light chains, these hybridomas (quadromas) produce a potential mixture of 10 different antibody molecules, of which only one has the correct bispecific structure. Purification of the correct molecule, which is usually done by affinity chromatography steps, is rather cumbersome, and the product yields are low. Similar procedures are disclosed in WO 93/08829, and in Traunecker et al., EMBO J, 10:3655-3659 (1991).

According to a different approach, antibody variable domains with the desired binding specificities (antibody-antigen combining sites) are fused to immunoglobulin constant domain sequences. The fusion preferably is with an immunoglobulin heavy chain constant domain, comprising at least part of the hinge, CH2, and CH3 regions. It is preferred to have the first heavy chain constant region (CH1) containing the site necessary for light chain binding, present in at least one of the fusions. DNAs encoding the immunoglobulin heavy chain fusions and, if desired, the immunoglobulin light chain, are inserted into separate expression vectors, and are co-transfected into a suitable host organism. This provides for great flexibility in adjusting the mutual proportions of the three polypeptide fragments in embodiments when unequal ratios of the three polypeptide chains used in the construction provide the optimum yields. It is, however, possible to insert the coding sequences for two or all three polypeptide chains in one expression vector when the expression of at least two polypeptide chains in equal ratios results in high yields or when the ratios are of no particular significance.

In a preferred embodiment of this approach, the bispecific antibodies are composed of a hybrid immunoglobulin heavy chain with a first binding specificity in one arm, and a hybrid immunoglobulin heavy chain-light chain pair (providing a second binding specificity) in the other arm. It was found that this asymmetric structure facilitates the separation of the desired bispecific compound from unwanted immunoglobulin chain combinations, as the presence of an immunoglobulin light chain in only one half of the bispecific molecule provides for a facile way of separation. This approach is disclosed in WO 94/04690. For further details of generating bispecific antibodies see, for example, Suresh et al., Methods in Enzymology, 121:210 (1986).

According to another approach described in U.S. Pat. No. 5,731,168, the interface between a pair of antibody molecules can be engineered to maximize the percentage of heterodimers that are recovered from recombinant cell culture. The preferred interface comprises at least a part of the C_{H}3 domain of an antibody constant domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers.

Bispecific antibodies include cross-linked or “heteroconjugate” antibodies. For example, one of the antibodies in the heteroconjugate can be coupled to avidin, the other to biotin. Such antibodies have, for example, been proposed to target immune system cells to unwanted cells (U.S. Pat. No. 4,676,980), and for treatment of HIV infection (WO 91/00360, WO 92/200373, and EP 03089). Heteroconjugate antibodies may be made using any convenient cross-linking methods. Suitable cross-linking agents are well known in the art, and are disclosed in U.S. Pat. No. 4,676,980, along with a number of cross-linking techniques.

Techniques for generating bispecific antibodies from antibody fragments have also been described in the literature. For example, bispecific antibodies can be prepared using chemical linkage. Brennan et al., Science, 229: 81 (1985) describe a procedure wherein intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Various techniques for making and isolating bispecific antibody fragments directly from recombinant cell culture have also been described. For example, bispecific antibodies have been produced using leucine zippers. Kostelny et al., J. Immunol., 148(5):1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA, 90:6444-6448 (1993) has provided an alternative mechanism for making bispecific antibody fragments. The fragments comprise a heavy chain variable domain (V_{H}) connected to a light chain variable domain (V_{L}) by a linker that is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See Gruber et al., J. Immunol., 152:5368 (1994).

Antibodies with more than two valencies are contemplated. For example, trispecific antibodies can be prepared. Tutt et al., J. Immunol. 147: 60 (1991).

(vii) Conjugates and Other Modifications of Antibodies

Modifications of the antibody are contemplated herein. Thus, in one embodiment, the antibody may be conjugated to another molecule, for example, to increase half-life or stability or otherwise improve the pharmacokinetics of the antibody. For example, the antibody may be linked to one of a variety of non-proteinaceous polymers, e.g., polyethylene glycol (PEG), polypropylene glycol, polyoxyalkylenes, or copolymers of polyethylene glycol and polypropylene glycol. Antibody fragments, such as Fab′, linked to one or more PEG molecules are an especially preferred embodiment of the invention.

The antibodies disclosed herein may also be formulated as liposomes. Liposomes containing the antibody are prepared by methods known in the art, such as described in Epstein et al., Proc. Natl. Acad. Sci. USA, 82:3688 (1985); Hwang et al., Proc. Natl. Acad. Sci. USA, 77:4030 (1980); U.S. Pat. Nos. 4,485,045 and 4,544,545; and WO 97/38731 published Oct. 23, 1997. Liposomes with enhanced circulation time are disclosed in U.S. Pat. No. 5,013,556.

Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter. Fab′ fragments of an antibody of the present invention can be conjugated to the liposomes as described in Martin et al. J. Biol. Chem. 257: 286-288 (1982) via a disulfide interchange reaction.

Amino acid sequence modification(s) of protein or peptide antibodies described herein are contemplated. For example, it may be desirable to improve the binding affinity and/or other biological properties of the antibody. Amino acid sequence variants of the antibody are prepared by introducing appropriate nucleotide changes into the antibody nucleic acid, or by peptide synthesis. Such modifications include, for example, deletions from, and/or insertions into and/or substitutions of, residues within the amino acid sequences of the antibody. Any combination of deletion, insertion, and substitution is made to arrive at the final construct, provided that the final construct possesses the desired characteristics. The amino acid changes also may alter post-translational processes of the antibody, such as changing the number or position of glycosylation sites.

A useful method for identification of certain residues or regions of the antibody that are preferred locations for mutagenesis is called “alanine scanning mutagenesis” as described by Cunningham and Wells, Science, 244:1081-1085 (1989). Here, a residue or group of target residues are identified (e.g., charged residues such as arg, asp, his, lys, and glu) and replaced by a neutral or negatively charged amino acid (most preferably alanine or polyalanine) to affect the interaction of the amino acids with antigen. Those amino acid locations demonstrating functional sensitivity to the substitutions then are refined by introducing further or other variants at, or for, the sites of substitution. Thus, while the site for introducing an amino acid sequence variation is predetermined, the nature of the mutation per se need not be predetermined. For example, to analyze the performance of a mutation at a given site, ala scanning or random mutagenesis is conducted at the target codon or region and the expressed antibody variants are screened for the desired activity.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue or the antibody fused to a polypeptide or polymer. Other insertional variants of the antibody molecule include the fusion to the N- or C-terminus of the antibody of an enzyme, or a polypeptide that increases the serum half-life of the antibody.

Another type of variant is an amino acid substitution variant. These variants have at least one amino acid residue in the antibody molecule replaced by different residue. The sites of greatest interest for substitutional mutagenesis of antibodies include the hypervariable regions, but FR alterations are also contemplated. Conservative substitutions are shown in the table below under the heading of “preferred substitutions”. If such substitutions result in a change in biological activity, then more substantial changes, denominated “exemplary substitutions” in the table below, or as further described below in reference to amino acid classes, may be introduced and the products screened.

TABLE 2

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; | Leu |
| | Phe; Norleucine | |
| Leu (L) | Norleucine; Ile; Val; | Ile |
| | Met; Ala; Phe | |
| Lys (K) | Arg; Gln; Asn | Arg |
| Met (M) | Leu; Phe; Ile | Leu |
| Phe (F) | Trp, Leu, Val, Ile, Ala, Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; | Leu |
| | Ala; Norleucine | |

Substantial modifications in the biological properties of the antibody are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Amino acids may be grouped according to similarities in the properties of their side chains (in A. L. Lehninger, in Biochemistry, second ed., pp. 73-75, Worth Publishers, New York (1975)):

(1) non-polar: Ala (A), Val (V), Leu (L), Ile (I), Pro (P), Phe (F), Trp (W), Met (M)

(2) uncharged polar: Gly (G), Ser (S), Thr (T), Cys (C), Tyr (Y), Asn (N), Gln (O)

(3) acidic: Asp (D), Glu (E)

(4) basic: Lys (K), Arg (R), His(H)

Alternatively, naturally occurring residues may be divided into groups based on common side-chain properties:

(1) hydrophobic: Norleucine, Met, Ala, Val, Leu, Ile;

(2) neutral hydrophilic: Cys, Ser, Thr, Asn, Gln;

(3) acidic: Asp, Glu;

(4) basic: H is, Lys, Arg;

(5) residues that influence chain orientation: Gly, Pro;

(6) aromatic: Trp, Tyr, Phe.

Non-conservative substitutions will entail exchanging a member of one of these classes for another class.

Any cysteine residue not involved in maintaining the proper conformation of the antibody also may be substituted, generally with serine, to improve the oxidative stability of the molecule and prevent aberrant crosslinking. Conversely, cysteine bond(s) may be added to the antibody to improve its stability (particularly where the antibody is an antibody fragment such as an Fv fragment).

A particularly preferred type of substitutional variant involves substituting one or more hypervariable region residues of a parent antibody. Generally, the resulting variant(s) selected for further development will have improved biological properties relative to the parent antibody from which they are generated. A convenient way for generating such substitutional variants is affinity maturation using phage display. Briefly, several hypervariable region sites (e.g. 6-7 sites) are mutated to generate all possible amino substitutions at each site. The antibody variants thus generated are displayed in a monovalent fashion from filamentous phage particles as fusions to the gene III product of M13 packaged within each particle. The phage-displayed variants are then screened for their biological activity (e.g. binding affinity) as herein disclosed. In order to identify candidate hypervariable region sites for modification, alanine scanning mutagenesis can be performed to identify hypervariable region residues contributing significantly to antigen binding. Alternatively, or in additionally, it may be beneficial to analyze a crystal structure of the antigen-antibody complex to identify contact points between the antibody and antigen. Such contact residues and neighboring residues are candidates for substitution according to the techniques elaborated herein. Once such variants are generated, the panel of variants is subjected to screening as described herein and antibodies with superior properties in one or more relevant assays may be selected for further development.

Another type of amino acid variant of the antibody alters the original glycosylation pattern of the antibody. Such altering includes deleting one or more carbohydrate moieties found in the antibody, and/or adding one or more glycosylation sites that are not present in the antibody.

Glycosylation of polypeptides is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-aceylgalactosamine, galactose, or xylose to a hydroxyamino acid, most commonly serine or threonine, although 5-hydroxyproline or 5-hydroxylysine may also be used.

Addition of glycosylation sites to the antibody is conveniently accomplished by altering the amino acid sequence such that it contains one or more of the above-described tripeptide sequences (for N-linked glycosylation sites). The alteration may also be made by the addition of, or substitution by, one or more serine or threonine residues to the sequence of the original antibody (for O-linked glycosylation sites).

Where the antibody comprises an Fc region, the carbohydrate attached thereto may be altered. For example, antibodies with a mature carbohydrate structure that lacks fucose attached to an Fc region of the antibody are described in US Patent Appl No US 2003/0157108 (Presta, L.). See also US 2004/0093621 (Kyowa Hakko Kogyo Co., Ltd). Antibodies with a bisecting N-acetylglucosamine (GlcNAc) in the carbohydrate attached to an Fc region of the antibody are referenced in WO 2003/011878, Jean-Mairet et al. and U.S. Pat. No. 6,602,684, Umana et al. Antibodies with at least one galactose residue in the oligosaccharide attached to an Fc region of the antibody are reported in WO 1997/30087, Patel et al. See, also, WO 1998/58964 (Raju, S.) and WO 1999/22764 (Raju, S.) concerning antibodies with altered carbohydrate attached to the Fc region thereof. See also US 2005/0123546 (Umana et al.) on antigen-binding molecules with modified glycosylation.

The preferred glycosylation variant herein comprises an Fc region, wherein a carbohydrate structure attached to the Fc region lacks fucose. Such variants have improved ADCC function. Optionally, the Fc region further comprises one or more amino acid substitutions therein which further improve ADCC, for example, substitutions at positions 298, 333, and/or 334 of the Fc region (Eu numbering of residues). Examples of publications related to “defucosylated” or “fucose-deficient” antibodies include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; Okazaki et al. J. Mol. Biol. 336:1239-1249 (2004); Yamane-Ohnuki et al. Biotech. Bioeng. 87: 614 (2004). Examples of cell lines producing defucosylated antibodies include Lec13 CHO cells deficient in protein fucosylation (Ripka et al. Arch. Biochem. Biophys. 249:533-545 (1986); US Patent Appl No US 2003/0157108 A1, Presta, L; and WO 2004/056312 A1, Adams et al., especially at Example 11), and knockout cell lines, such as alpha-1,6-fucosyltransferase gene, FUT8, knockout CHO cells (Yamane-Ohnuki et al. Biotech. Bioeng. 87: 614 (2004)).

Nucleic acid molecules encoding amino acid sequence variants of the antibody are prepared by a variety of methods known in the art. These methods include, but are not limited to, isolation from a natural source (in the case of naturally occurring amino acid sequence variants) or preparation by oligonucleotide-mediated (or site-directed) mutagenesis, PCR mutagenesis, and cassette mutagenesis of an earlier prepared variant or a non-variant version of the antibody.

It may be desirable to modify the antibody of the invention with respect to effector function, e.g. so as to enhance ADCC and/or CDC of the antibody. This may be achieved by introducing one or more amino acid substitutions in an Fc region of an antibody. Alternatively or additionally, cysteine residue(s) may be introduced in the Fc region, thereby allowing interchain disulfide bond formation in this region. The homodimeric antibody thus generated may have improved internalization capability and/or increased complement-mediated cell killing and ADCC. See Caron et al., J. Exp Med. 176:1191-1195 (1992) and Shopes, J. Immunol. 148:2918-2922 (1992). Homodimeric antibodies may also be prepared using heterobifunctional cross-linkers as described in Wolff et al. Cancer Research 53:2560-2565 (1993). Alternatively, an antibody can be engineered that has dual Fc regions and may thereby have enhanced complement lysis and ADCC capabilities. See Stevenson et al. Anti-Cancer Drug Design 3:219-230 (1989).

WO 00/42072 (Presta, L.) describes antibodies with improved ADCC function in the presence of human effector cells, where the antibodies comprise amino acid substitutions in the Fc region thereof. Preferably, the antibody with improved ADCC comprises substitutions at positions 298, 333, and/or 334 of the Fc region. Preferably the altered Fc region is a human IgG1 Fc region comprising or consisting of substitutions at one, two or three of these positions.

Antibodies with altered C1q binding and/or CDC are described in WO 99/51642, U.S. Pat. No. 6,194,551B1, U.S. Pat. No. 6,242,195B1, U.S. Pat. No. 6,528,624B1 and U.S. Pat. No. 6,538,124 (Idusogie et al.). The antibodies comprise an amino acid substitution at one or more of amino acid positions 270, 322, 326, 327, 329, 313, 333 and/or 334 of the Fc region thereof.

To increase the serum half-life of the antibody, one may incorporate a salvage receptor binding epitope into the antibody (especially an antibody fragment) as described in U.S. Pat. No. 5,739,277, for example. As used herein, the term “salvage receptor binding epitope” refers to an epitope of the Fc region of an IgG molecule (e.g., IgG1, IgG₂, IgG₃, or IgG4) that is responsible for increasing the in vivo serum half-life of the IgG molecule. Antibodies with substitutions in an Fc region thereof and increased serum half-lives are also described in WO00/42072 (Presta, L.).

Engineered antibodies with three or more (preferably four) functional antigen binding sites are also contemplated (US Appln No. US 2002/0004587 A1, Miller et al.).

### IV. Pharmaceutical Formulations

Exemplary anti-CD20 antibody formulations are described in WO98/56418, expressly incorporated herein by reference. Another formulation is a liquid multidose formulation comprising the anti-CD20 antibody at 40 mg/mL, 25 mM acetate, 150 mM trehalose, 0.9% benzyl alcohol, 0.02% polysorbate 20 at pH 5.0 that has a minimum shelf life of two years storage at 2-8° C. Another anti-CD20 formulation of interest comprises 10 mg/mL antibody in 9.0 mg/mL sodium chloride, 7.35 mg/mL sodium citrate dihydrate, 0.7 mg/mL polysorbate 80, and Sterile Water for Injection, pH 6.5. Yet another aqueous pharmaceutical formulation comprises 10-30 mM sodium acetate from about pH 4.8 to about pH 5.5, preferably at pH5.5, polysorbate as a surfactant in a an amount of about 0.01-0.1% v/v, trehalose at an amount of about 2-10% w/v, and benzyl alcohol as a preservative (U.S. Pat. No. 6,171,586). Lyophilized formulations adapted for subcutaneous administration are described in WO97/04801. Such lyophilized formulations may be reconstituted with a suitable diluent to a high protein concentration and the reconstituted formulation may be administered subcutaneously to the mammal to be treated herein.

One formulation for the humanized 2H7 variants is antibody at 12-14 mg/mL in 10 mM histidine, 6% sucrose, 0.02% polysorbate 20, pH 5.8.

In a specific embodiment, 2H7 variants and in particular Variant A is formulated at 20 mg/mL antibody in 10 mM histidine sulfate, 60 mg/ml sucrose., 0.2 mg/ml polysorbate 20, and Sterile Water for Injection, at pH5.8.

The formulation herein may also contain more than one active compound as necessary for the particular indication being treated, preferably those with complementary activities that do not adversely affect each other. For example, it may be desirable to further provide a cytotoxic agent, chemotherapeutic agent, cytokine or immunosuppressive agent (e.g. one which acts on T cells, such as cyclosporin or an antibody that binds T cells, e.g. one which binds LFA-1). The effective amount of such other agents depends on the amount of antibody present in the formulation, the type of disease or disorder or treatment, and other factors discussed above. These are generally used in the same dosages and with administration routes as described herein or about from 1 to 99% of the heretofore employed dosages.

The formulations to be used for in vivo administration must be sterile. This is readily accomplished by filtration through sterile filtration membranes.

### V. Articles of Manufacture

In another embodiment of the invention, articles of manufacture containing materials useful for the treatment of autoimmune diseases are provided. The invention, in particular, provides an article of manufacture comprising: (a) a container comprising an antagonist such as an antibody that binds to a B-cell surface marker (e.g., a CD20 antibody) (preferably the container comprises the antibody and a pharmaceutically acceptable carrier or diluent within the container); and (b) a package insert with instructions for treating an autoimmune disease, such as rheumatoid arthritis in a subject, wherein the instructions indicate that the subject is administered the complete therapeutically effective amount of the antagonist or antibody (e.g., CD20 antibody) as single intravenous infusion. In one

In a particular embodiment, the article of manufacture herein further comprises a container comprising a second medicament, wherein the antagonist or antibody is a first medicament, and which article further comprises instructions on the package insert for treating the subject with the second medicament, in an effective amount. The second medicament may be any of those set forth above, with an exemplary second medicament being those set forth above, including an antibiotic, an immunosuppressive agent, a disease-modifying anti-rheumatic drug (DMARD), a pain-control agent, an integrin antagonist, a non-steroidal anti-inflammatory drug (NSAID), a cytokine antagonist, bisphosphonate, or a hormone, or a combination thereof, more preferably a DMARD, NSAID, pain-control agent, or immunosuppressive agent. Most preferably, the second medicament is methotrexate.

In this aspect, the package insert is on or associated with the container. Suitable containers include, for example, bottles, vials, syringes, etc. The containers may be formed from a variety of materials such as glass or plastic. The container holds or contains a composition that is effective for treating the target autoimmune disease and may have a sterile access port (for example the container may be an intravenous solution bag or a vial having a stopper pierceable by a hypodermic injection needle). At least one active agent in the composition is the CD20 antagonist, such as a CD20 antibody. The article of manufacture may further comprise an additional container comprising a pharmaceutically acceptable diluent buffer, such as bacteriostatic water for injection (BWFI), phosphate-buffered saline, Ringer's solution, and/or dextrose solution. The article of manufacture may further include other materials desirable from a commercial and user standpoint, including other buffers, diluents, filters, needles, and syringes.

Further details of the invention will be apparent from the following non-limiting Examples.

### Reference Example

The humanized 2H7 antibody variants were prepared and assayed for biological function including human CD20 binding affinity, effector functions and B cell depletion were as described in WO 04/056312, incorporated herein by reference in its entirety. The murine 2H7 antibody variable region sequences and the chimeric 2H7 with the mouse V and human C have been described, see, e.g., U.S. Pat. Nos. 5,846,818 and 6,204,023, incorporated herein by reference in their entirety.

### Example 1

### Clinical Phase I/II Trial to Study the Efficacy and Safety of Ocrelizumab Administered as a Single Intravenous Infusion to Patients with Active Rheumatoid Arthritis

The efficacy of ocrelizumab (OCR), a CD20 B cell-targeted humanized monoclonal antibody, administered as a single intravenous (i.v.) infusion, was tested on ACR (American College of Rheumatology) responses in patients with moderate to severe rheumatoid arthritis (RA) receiving concomitant methotrexate (MTX) at a stable dose (10-25 mg/week) after failure of one to six DMARDS (including biologics).

In particular, a randomized placebo-controlled, multicenter, Phase I/II study of the safety of escalating single intravenous doses of rhuMAb 2H7 (Ro 496-4913, PRO70769) was conducted in patients with moderate to severe rheumatoid arthritis receiving stable doses of concomitant methotrexate but with unsatisfactory clinical response. The objective of the trial was to evaluate the safety and tolerability in combination with methotrexate (MTX) in patients with moderate to severe rheumatoid arthritis (RA) of escalating single intravenous (IV) doses of rhuMAb 2H7.

The trial was conducted using the following protocol:

### Part I—Dose Escalation

Patients receive a single IV infusion of rhuMAb 2H7 or placebo equivalent at one of the following dose levels:

Group 1: 400 mg

Group 2: 1000 mg

Group 3: 1500 mg

Dose escalation occurs after the treatment of a minimum of 10 patients per dose level (8 active; 2 placebo): The decision to escalate is based on a review of the data from the previous group within the study. The number of dose-limiting toxicities will be assessed and depending on this number, the study may proceed to the next dose level or additional patients may be enrolled at the same dose level.

### Part II

After the dose escalation stage and an interim analysis of the data, an additional 120 patients (96 active, 24 placebo) will be randomized to three of the doses used in Part I (anticipated to be 400, 1000 and 1500 mg), provided that these have been demonstrated to be tolerable during the dose escalation stage.

### Number of Patients

At least 40 in Part I; 120 in Part II

### Target Population

Adult patients (aged 18-80 years)
Moderate to severe R^{A}
Failed (through lack of efficacy or tolerability) one disease-modifying antirheumatic drug (DMARD) or biologic, but have not failed with more than five of these agents
Currently receiving MTX, but have unsatisfactory clinical responses to treatment with MTX (i.e. partial responders)

### Length of Study

Single IV infusion; assessments to 24 weeks followed by a further 48 week follow-up: total 72 weeks
Patients who withdraw at any time or who are still B-cell depleted at Week 72 will enter a safety follow-up for 48 weeks or until their B-cells have recovered (whichever is longer).

### Significant Inclusion Criteria

Have active RA:
Diagnosis of RA for at least 6 months (according to revised 1987 ACR criteria)
Positive serum rheumatoid factor (≧20 IU/L)
C-reactive protein ≧1.5 mg/dL or erythrocyte sedimentation rate ≧28 mm/hr at screening
Swollen joint count of ≧8 (66 joint count) and tender joint count of ≧8 (68 joint count) at screening and randomization

### Previous Treatment/Background Treatments:

Current treatment on outpatient basis
Current treatment with MTX at 10-25 mg weekly for at least 12 weeks, stable for the last 4 weeks prior to screening
All DMARDs (except MTX) withdrawn 4-8 weeks before randomization; 8 weeks for etanercept, infliximab, adalimumab and leflunomide (after cholestyramine drug washout in those who received leflunomide)
Corticosteroid dose not to exceed 10 mg/day prednisone or equivalent, stable for the last 4 weeks prior to screening
If receiving NSAIDs, dose must be stable 2 weeks prior to screening.
Willing to receive oral folic acid

### Investigational Product(s)

Dose—Single doses of rhuMAb 2H7 at 400, 1000, 1500 and 2000 mg.

Route —IV infusion (Rates provided in Appendix 3 to the Protocol)

### Comparator “Drug” Dose/Route/Regimen

Single placebo infusion

### Concomitant Treatments

Peri-Infusion Treatment

Prophylaxis (recommended)—Prophylactic does of acetaminophen and diphenhydramine (or other non-sedating antihistamine), give 30-60 minutes before the infustion of rhuMAB 2H7 is initiated, are recommended.

Possible steroid cover during infusion—If significant infusion-related events are observed at a given dose level, and a reduction in the infusion rate does not reduce the number of infusion-related events, the Sponsor may recommend premedication with IV corticosteroids for patients treated subsequently.

Methotrexate (required)—At a stable weekly dose of 10-25 mg (oral or parenteral); provided by the study center's pharmacy

Corticosteroids (optinal)—Continue at stable oral doses of up to 10 mg/day prednisone or prednisone equivalent

NSAIDs—Continue at stable doses

### Re-Treatment

It is expected that the onset of clinical response may be slow. Therefore, when possible, background treatment should be kept unchanged until Week 24. After Week 24, eligible patients may receive re-treatment under a separate re-treatment protocol

### How Supplied

rhuMAb 2H7 and matching placebo will be supplied by the Sponsor in 10 mL vials; rhuMAb 2H7 will be at a concentration of 20 mg/mL. rhuMAb 2H7 must be diluted in 0.9% sodium chloride before infusion, to provide the appropriate dose in a volume of 250 or 500 mL (dilution information is provided in Appendix 3 to the Protocol)

Other medications will be prescribed by the Investigator and provided locally by the center pharmacy.

### Assessments of:

Safety

Adverse Events
Incidence of adverse events (nature, intensity, seriousness, relationship, reversibility and treatment; graded using NCl CTCAE v3.0) to include infusion-related reactions and dose limiting toxicities (see Procedures section for definitions)

Hematology Clinical Chemistry and Utinalysis
Incidence of clinical laboratory abnormalites

Immunology
Incidence of human anti-rhuMAB 2H7 antibodies
Rheumatoid factor concentrations
Serum immunoglobulin concentrations (IgG, IgM, IgA, IgE)
Serum concentration of tetanus toxoid antibodies
Peripheral B-cell counts; T-cell counts

Efficacy
Percentage of patients with clinical responses of 20%, 50% and 70% according to ACR criteria and the components of this outcome measure at Week 24, using Day 1 pre-infusion ACR core set values as baseline.
Disease activity score and proportion of EULAR responders at Week 24

### Statistical Analyses:

All analyses will be exploratory in nature. Patients will be grouped according to treatment actually received. All patients who receive any study drug (rhuMAb 2H7 or placebo) will be included for purposes of analysis.

Patients who, during the first 24 weeks of the study, require an increase in MTX dose, corticosteroid dose, introduction of an additional intervention or excluded medication will be considered non-responders in the efficacy analysis.

Control (placebo) patients at the different dose levels will be combined into one control group. All results will be presented separately for the control group and each of the active dose groups: 400 mg, 1000 mg, 1500 mg and 2000 mg.

Two subgroup analyses will be conducted: one based on all enrolled patients, and the other based on those patients who are randomized in Part II of the study after completion of dose escalation in Part I.

Pharmacokinetics:

Serum concentrations of rhuMAb 2H7 and computed pharmacokinetic parameters will be listed by patient and dose group and summarized descriptively (mean, standard deviation, percent coefficient of variation, minimum, maximum). Individual and mean concentration versus time plots will be presented on both linear and logarithmic scales. Pharmacokinetic parameters will be computed using non-compartmental methods.

In all calculations, concentrations below the limit of quantification of the assay will be deleted or assigned a numeric value based on the lower reporting limit of the assay.

Pharmacodynamics:

The B-cell depletion profile for each patient will be determined and summarized descriptively for each dose level. The pharmacodynamics of peripheral B-cell depletion and repletion will be characterized by the extent and duration of depletion at each dose level. In addition, a prospective analysis plan will be prepared for providing population pharmacokinetic modeling as a pharmacokinetic/pharmacodynamic analysis of the relationship between the pharmacokinetics of rhuMAb 2H7 and peripheral B-cell depletion and repletion.

Formulation:

rhuMAb 2H7 drug product is manufactured by Genentech, as a sterile, clear, colorless, preservative free liquid intended for dilution for IV administration. The rhuMAb 2H7 drug product is supplied at a concentration of 20 mg/mL in 10 cc single-use vials, which contain a nominal 10 mL (200 mg) of rhuMAb 2H7 per vial. The rhuMAb 2H7 drug product is formulated in 10 mM histidine sulfate, 60 mg/mL sucrose, 0.2 mg/mL polysorbate 20, and Sterile Water for Injection. The pH is adjusted to 5.8.

rhuMAb 2H7 drug product must be diluted before administration. Solutions of rhuMAb 2H7 for IV administration are prepared by dilution of the drug product into an infusion bag containing 0.9% sodium chloride, to a final drug concentration of 1.6-6 mg/mL. rhuMAb 2H7-matching placebo is also supplied in 10 cc single-use vials that contain a nominal 10 mL of placebo solution per vial. It is identical in composition to the rhuMAb 2H7 drug product but does not contain rhuMAb 2H7. rhuMAb 2H7 placebo solutions for IV administration are prepared by dilution of the rhuMAb 2H7 placebo into infusion bags containing 0.9% sodium chloride, using an identical procedure as for the rhuMAb 2H7 drug product.

### Dosage, Administration:

Study drug should be given as a slow IV infusion at the rates shown. The rates are dependent on the dose to be administered and will not exceed 250 mL/hr. It should not be administered as an IV push or bolus. Study drug infusions should be made through a dedicated line. Isotonic 0.9% sodium chloride solution should be used as an infusion vehicle. It is recommended that patients receive prophylactic treatment with acetaminophen (1 g) and diphenhydramine HCl (50 mg; or equivalent dose of similar agent) by mouth 30-60 minutes prior to the start of an infusion. All doses except 2000 mg will be administered in a volume of 250 mL; the 2000 mg dose (and matching placebo) will be administered in a volume of 500 mL.

### Retreatment Eligibility

Re-treatment for patients in whom symptoms recur may be given after Week 24 only to those patients who received active treatment and have achieved at least an ACR20 response at Week 24 after receiving their initial treatment, and who did not experience significant adverse events during the study. Retreatment for these patients will be at the same dose as their initial regimen if symptoms recur (indicated by SJC≧8 and TJC≧8 after Week 24).

### Infusion Schedules

The rhuMAb 2H7 drug product is supplied as a liquid at a concentration of 20 mg/mL in 10 cc single-use vials, which contain a nominal 10 mL (200 mg) of rhuMAb 2H7 per vial. Matching placebo, identical in composition but not containing rhuMAb 2H7 is also supplied as a liquid in 10 cc single use vials, which contain a nominal 10 mL of placebo solution per vial.

The rituximab (MabThera) prescribing information provides the following information on infusion:

First Infusion: The recommended initial rate for infusion is 50 mg/hr; after the first 30 minutes, it can be escalated in 50 mg/hr increments every 30 minutes to a maximum of 400 mg/hr. If hypersensitivity or an infusion related event develops, the infusion should be temporarily slowed or interrupted and can continue at one half the previous rate upon improvement of symptoms. If the same adverse reactions occur severely for a second time the decision to stop treatment should be considered. The infusion rate may be increased upon improvement of symptoms.

Subsequent Infusions: Infuse at initial rate of 100 mg/hr and increase by 100 mg/hr increments at 30 minute intervals, to a maximum of 400 mg/hr.

The ongoing ACT2847g study uses the same standard infusion rates, with their highest dose of 1000 mg escalating to 400 mg/hr, with an overall infusion time of approximately 4 hr 15 min.

In order to investigate faster infusion times, this protocol will use the following schedules, though if there is any indication from the lowest dose group in Part I that faster infusion rates are not tolerated, they will be reduced and the incremental rate of infusion in mg/hr reduced to the standard rate of 50 mg/hr steps for subsequent dose groups. 400 mg dose group, at concentration of 1.6 mg/mL; total infusion volume 250 mL

| | Infusion | Infusion | Dose (mg) | Cumulative dose in |
|---|---|---|---|---|
| Time | rate | rate | in 30 min | mg |
| (minutes) | mg/hr | mL/hr | (volume) | (cumulative volume) |
| 0-30 | 50 | 31.25 | 25 (15.63) | 25 (15.63) |
| 31-60 | 100 | 62.5 | 50 (31.25) | 75 (46.88) |
| 61-90 | 200 | 125 | 100 (62.5) | 175 (109.38) |
| 91-120 | 400 | 250 | 200 (125) | 375 (234.38) |
| Remaining | 400 | 250 | Remaining 25 mg | 400 (250) |
| (approx 5 | | | | |

Approximate total infusion time = 2 hours 5 minutes

1000 mg dose group, at concentration of 4 mg/mL;
total infusion volume 250 mL

| | Infusion | | Dose (mg) | |
|---|---|---|---|---|
| Time | rate | Infusion | in 30 min | Cumulative dose in |
| (minutes) | mg/hr | mL/hr | (volume) | (cumulative volume) |
| 0-30 | 50 | 12.5 | 25 (6.25) | 25 (6.25) |
| 31-60 | 100 | 25 | 50 (12.5) | 75 (18.75) |
| 61-90 | 200 | 50 | 100 (25) | 175 (43.75) |
| 91-120 | 400 | 100 | 200 (50) | 375 (93.75) |
| 121-150 | 600 | 150 | 300 (75) | 675 (168.75) |
| 151-180 | 600 | 150 | 300 (75) | 975 (243.75) |
| Remaining | 600 | 150 | Remaining 25 mg | 1000 (250) |
| (approx 3 | | | | |

Approximate total infusion time = 3 hours 3 minutes

1500 mg dose group, at concentration of 6 mg/ml;
total infusion volume 250 mL

| | Infusion | | Dose (mg) | |
|---|---|---|---|---|
| Time | rate | Infusion | in 30 min | Cumulative dose in |
| (minutes) | mg/hr | mL/hr | (volume) | (cumulative volume) |
| 0-30 | 50 | 8.3 | 25 (4.15) | 25 (4.15) |
| 31-60 | 100 | 16.7 | 50 (8.35) | 75 (12.5) |
| 61-90 | 200 | 33.3 | 100 (16.7) | 175 (29.2) |
| 91-120 | 400 | 66.7 | 200 (33.3) | 375 (62.5) |
| 121-150 | 600 | 100 | 300 (50) | 675 (112.5) |
| 151-180 | 800 | 133 | 400 (66.7) | 1075 (179.2) |
| 181-210 | 800 | 133 | 400 (66.7) | 1475 (245.9) |
| Remaining | 800 | 133 | Remaining 25 mg | 1500 (250) |
| (approx 2 | | | | |

Approximate total infusion time = 3 hours 32 minutes

2000 mg dose group, at concentration of 4 mg/mL;
total infusion volume 500 mL

| | | | Dose (mg) | |
|---|---|---|---|---|
| Time | Infusion rate | Infusion rate | in 30 min | Cumulative |
| (minutes) | mg/hr | mL/hr | (volume in mL) | dose in mg |
| 0-30 | 50 | 12.5 | 25 (6.25) | 25 (6.25) |
| 31-60 | 100 | 25 | 50 (12.5) | 75 (18.75) |
| 61-90 | 200 | 50 | 100 (25) | 175 (43.75) |
| 91-120 | 400 | 100 | 200 (50) | 375 (93.75) |
| 121-150 | 600 | 150 | 300 (75) | 675 (168.75) |
| 151-180 | 800 | 200 | 400 (100) | 1075 (268.75) |
| 181-210 | 800 | 200 | 400 (100) | 1475 (368.75) |
| 211-240 | 800 | 200 | 400 (100) | 1875 (468.75) |
| Remaining | 800 | 200 | Remaining | 2000 (500) |
| (approx 10 | | | 125 mg (32) | |

Approximate total infusion time = 4 hours 10 minutes

Active RA was defined by ≧8 swollen and ≧8 tender joints and elevated C-reactive protein (CRP) or erythrocyte sedimentation rate (ESR). Patients (n=175) were randomized to receive single intravenous infusions of either OCR (400, 1000, 1500, or 2000 mg) or matching placebo (PLO). Forty patients participated in a 4 dose escalation part I of this study (2 placebo, 8 active in each cohort) and 135 other patients randomized to the lower three doses or PLO were included in the safety and ITT analyses. No peri-infusional glucocorticoids were given prior to study drug (OCR). Clinical assessments were conducted at 2-weekly intervals up to week 8 and then 4 weekly to week 24 The primary endpoint was the proportion of patients that achieved an ACR20 response at week 24

### Results

70% and 89% of the OCR and PLO groups were female and, at baseline, the mean numbers of tender and swollen joints were 16 & 17 and 28 & 28, ESR 45 & 49, CRP 25 & 21, RF+92% & 94% respectively. B cell depletion (96% for OCR at week 12) and clinical responses were observed in all OCR dose groups ACR20/50/70 responses at week 24 were 57%, 31% and 15% for the combined OCR group and 40%, 10% and 3% for PLO group respectively.

| | Placebo | 400 mg | 1000 mg | 1500 mg | 2000 mg |
|---|---|---|---|---|---|
| Safety results | N = 35 | N = 43 | N = 44 | N = 45 | N = 8 |
| Adverse events during infusion | 4 (11%) | 18 (42%) | 25 (57%) | 21 (47%) | 2 (25%) |
| Infection rate/patient year | 1.3 | 0.9 | 0.9 | 0.8 | 1.9 |
| Completed 24 weeks | 30 (86%) | 42 (98%) | 41 (91%) | 41 (91%) | 8 (100%) |
| Withdrew (WD) | 5 (14%) | 1 (2%) | 4 (9%) | 4 (9%) | — |
| WD due to intolerance | — | — | 1 (2%) | 1 (2%) | — |
| WD due to insufficient response | 5 (14%) | 1 (2%) | 3 (7%) | 3 (7%) | — |

### CONCLUSIONS

Treatment with single infusions of the humanized anti CD20 ocrelizumab and no peri-infusional steroids in RA patients receiving background MTX achieved B cell depletion at all doses and was accompanied by increased ACR20/50/70 responses versus MTX alone Single infusions were well tolerated with only 2 withdrawals due to intolerance. The incidence and type of infections was similar in the PLO and OCR groups.

The foregoing written specification is considered to be sufficient to enable one skilled in the art to practice the invention. The present invention is not to be limited in scope by the construct deposited, since the deposited embodiment is intended as a single illustration of certain aspects of the invention and any constructs that are functionally equivalent are within the scope of this invention. The deposit of material herein does not constitute an admission that the written description herein contained is inadequate to enable the practice of any aspect of the invention, including the best mode thereof, nor is it to be construed as limiting the scope of the claims to the specific illustrations that it represents. Indeed, various modifications of the invention in addition to those shown and described herein will become apparent to those skilled in the art from the foregoing description and fall within the scope of the appended claims.

From the description of the invention herein, it is manifest that various equivalents can be used to implement the concepts of the present invention without departing from its scope. Moreover, while the invention has been described with specific reference to certain embodiments, a person of ordinary skill in the art would recognize that changes can be made in form and detail without departing from the spirit and the scope of the invention. The described embodiments are considered in all respects as illustrative and not restrictive. It should also be understood that the invention is not limited to the particular embodiments described herein, but is capable of many equivalents, rearrangements, modifications, and substitutions without departing from the scope of the invention. Thus, additional embodiments are within the scope of the invention and within the following claims.

All U.S. patents and applications; foreign patents and applications; scientific articles; books; and other publications mentioned herein are hereby incorporated by reference in their entirety as if each individual document was specifically and individually indicated to be incorporated by reference, including any drawings, figures and tables, as though set forth in full.

What is claimed is:

1. A method of depleting B cells in a human patient having an autoimmune disease comprising administering to the patient a complete therapeutically effective amount of a CD20 specific antagonist by one intravenous (i.v.) infusion.

2. The method of claim 1 further comprising administering a second i.v. infusion of a complete therapeutically effective amount of the CD20 specific antagonist 4 to 6 months after first adminstering said complete therapeutically affective amount of the CD20 specific antagonist.

3. The method of claim 2 wherein the second i.v. infusion is administered to a patient who responds to but relapses following receipt of the complete therapeutically affective amount of a CD20 antagonist.

4. The method of claim 1 wherein the CD20 specific antagonist is a CD20 specific monoclonal antibody.

5. The method of claim 1 wherein the CD20 specific antagonist is a CD20 specific monoclonal antibody.

6. The method of claim 2 wherein the therapeutically effective amounts of said first and second i.v. infusions are essentially the same.

7. The method of claim 4 wherein the CD20 specific monoclonal antibody is chimeric, humanized or human.

8. The method of claim 4 wherein the autoimmune disease is selected from the group consisting of rheumatoid arthritis, systemic lupus erythematosus (SLE), lupus nephritis, ulcerative colitis, Crohn's disease, Sjorgen's syndrome, neuormyelitis optica (NMO), ANCA associated-vasculitis, Wegener's disease, inflammatory bowel disease, idiopathic thrombocytopenic purpura (ITP), thrombotic thrombocytopenic purpura (TTP), autoimmune thrombocytopenia, multiple sclerosis, psoriasis, IgA nephropathy, IgM polyneuropathies, myasthenia gravis, diabetes mellitus, Reynaud's syndrome, and glomerulonephritis.

9. The method of claim 8 wherein the autoimmune disease is rheumatoid arthritis.

10. The method of claim 9 wherein the patient has shown inadequate response to prior treatment with methotrexate, a TNF antagonist, or a different CD20 antagonist.

11. The method of claim 9 wherein the autoimmune disease is active rheumatoid arthritis.

12. The method of claim 11 wherein the active rheumatoid arthritis is moderate to severe rheumatoid arthritis.

13. The method of claim 10 wherein the therapeutically effective amount of said CD20 specific antagonist is between 10 mg and 2000 mg.

14. The method of claim 13 wherein the CD20 specific antibody is a humanized 2H7 antibody.

15. The method of claim 14 wherein the therapeutically effective amount of said CD20 specific antagonist is between 400 mg and 1500 mg.

16. The method of claim 15 wherein the therapeutically effective amount of said CD20 specific antagonist is 400 mg.

17. The method of claim 15 wherein the therapeutically effective amount of said CD20 specific antagonist is 1000 mg.

18. The method of claim 15 wherein the therapeutically effective amount of said CD20 specific antagonist is 1500 mg.

19. The method of claim 14 wherein the humanized CD20 specific antibody is selected from the group consisting of 2H7 Variants A, B and I, comprising a full length L chain of SEQ ID NO: 6 and a full length heavy chain of SEQ ID NO: 7, 8 or 15, or a fragment thereof.

20. The method of claim 14 wherein the humanized CD20 specific antibody is 2H7 Variant A comprising a full length L chain of SEQ ID NO: 6 and a full length H chain of SEQ ID NO: 7, or a fragment thereof.

21. The method of claim 14 wherein the humanized CD20 specific antibody binds to the same epitope as 2H7 Variant A and comprises a full length L chain of SEQ ID NO: 6 and a full length H chain of SEQ ID NO: 7, or a fragment thereof.

22. The method of claim 14 wherein the humanized CD20 specific antibody is selected from the group consisting of 2H7 Variants C, D, F, G, and H comprising a full length L chain of SEQ ID NO: 9 and a full length H chain of SEQ ID NO: 10, 11, 12, 13, or 14, or a fragment thereof.

23. The method of claim 14 wherein the humanized CD20 specific antibody is 2H7 Variant B comprising a full length L chain of SEQ ID NO: 6 and a full length H chain of
SEQ ID NO: 8, or a fragment thereof.

24. The method of claim 14 wherein the humanized CD20 specific antibody is 2H7 Variant C comprising a full length L chain of SEQ ID NO: 9 and a full length H chain of SEQ ID NO: 10, or a fragment thereof.

25. The method of claim 14, wherein the CD20 specific antibody is Rituximab.

26. The method of claim 14, wherein the CD20 specific antibody is HUMAX-CD20™.

27. The method of claim 14, wherein the CD20 specific antibody is administered in conjunction with a therapeutic regime which comprises administering a drug selected from the group consisting of a nonsteroidal anti-inflammatory drug (NSAIDs), an analgesic, a glucocorticosteroid, cyclophosphamide, adalimumab, leflunomide, infliximab, etanercept, ofatumumab, tocilizumab, AME-133, lmmu-106, and a COX-2 inhibitor.

28. The method of claim 14, further comprising administering to the patient a second therapeutic agent.

29. The method of claim 28, wherein the second therapeutic agent is an immunosuppressive agent.

30. The method of claim 14, further comprising concomitant methotrexate treatment.

31. The method of claim 30 wherein said methotrexate is administered at a dose of from 10 mg to 25 mg/week.

32. The method of claim 30 wherein the patient has received one to six DMARDS prior to administration of said CD20 specific antibody.

33. The method of claim 14 wherein the patient did not receive any steroid treatment prior to the administration of the CD20 specific antibody.

34. A method for the treatment of active rheumatoid arthritis (RA) in a human patient comprising administering to the patient from 400 mg to 1500 mg of a CD20 specific antibody as a single intravenous (i.v.) infusion.

35. The method of claim 34 wherein the CD20 specific antibody is a chimeric, humanized or human monoclonal antibody.

36. The method of claim 35 wherein the humanized CD20 specific antibody is selected from the group consisting of 2H7 Variants A, B and I, comprising a full length L chain of SEQ ID NO: 6 and a full length heavy chain of SEQ ID NO: 7, 8 or 15, or a fragment thereof.

37. The method of claim 35 wherein the humanized CD20 specific antibody is 2H7 Variant A comprising a full length L chain of SEQ ID NO: 6 and a full length H chain of SEQ ID NO: 7, or a fragment thereof.

38. The method of claim 35 wherein the humanized CD20 specific antibody binds to the same epitope as 2H7 Variant A comprising a full length L chain of SEQ ID NO: 6 and a full length H chain of SEQ ID NO: 7, or a fragment thereof.

39. The method of claim 35 wherein the humanized CD20 specific antibody is selected from the group consisting of 2H7 Variants C, D, F, G, and H comprising a full length L chain of SEQ ID NO: 9 and a full length H chain of SEQ ID NO: 10, 11, 12, 13, or 14, or a fragment thereof.

40. The method of claim 35 wherein the humanized CD20 specific antibody is 2H7 Variant B comprising a full length L chain of SEQ ID NO: 6 and a full length H chain of SEQ ID NO: 8, or a fragment thereof.

41. The method of claim 35 wherein the humanized CD20 specific antibody is 2H7 Variant C comprising a full length L chain of SEQ ID NO: 9 and a full length H chain of SEQ ID NO: 10, or a fragment thereof.

42. The method of claim 35 wherein the CD20 specific antibody is Rituximab.

43. The method of claim 34 further comprising no administration of a CD20 specific antibody for at least 4 months.

44. The method of claim 34 comprising administering 400 mg of said CD20 specific antibody to said patient.

45. The method of claim 34 comprising administering 1000 mg of said CD20 specific antibody to said patient.

46. The method of claim 34 comprising administering 1500 mg of said CD20 specific antibody to said patient.

47. A method for the treatment of active rheumatoid arthritis (RA) in a human patient comprising administering from 400 mg to 1500 mg of 2H7 Variant A comprising a full length L chain of SEQ ID NO: 6 and a full length H chain of SEQ ID NO: 7, or a fragment thereof to said human patient.

48. The method of claim 47 in a human patient comprising administering 1000 mg of CD20 specific antibody 2H7 to said patient.

49. The method of claim 47, comprising administering 1500 mg of said CD20 specific antibody to said human patient.

50. The method of claim 47, comprising administering 400 mg of said CD20 specific antibody to said human patient.

51. The method of claim 47, wherein said administration is not followed by a second administration of a CD20 antibody for at least 4 months.

52. An article of manufacture comprising: (a) a container containing a CD20 specific antagonist; and (b) a package insert with instructions for treating an autoimmune disease in a human subject, wherein the instructions indicate that the subject receives the complete therapeutically effective amount of the CD20 specific antagonist as single intravenous infusion.

53. The article of manufacture of claim 52 wherein the CD20 antagonist is a chimeric, humanized or human CD20 specific monoclonal antibody.

54. The article of manufacture of claim 53 wherein the autoimmune disease is rheumatoid arthritis.

55. The article of manufacture of claim 54 wherein the CD20 specific antagonist is CD20 specific antibody 2H7 Variant A comprising a full length L chain of SEQ ID NO: 6 and a full length H chain of SEQ ID NO: 7, or a fragment thereof.

56. The article of manufacture of claim 55 wherein (a) contains between 400 mg and 1500 mg of CD20 specific antagonist.

57. An article of manufacture comprising: (a) a container comprising a CD20 specific antibody; and (b) a package insert with instructions for treating rheumatoid arthritis in a human subject, wherein the instructions indicate that the subject receives a complete therapeutically effective amount between 400 mg and 1500 mg of a CD20 specific antibody as single intravenous infusion.

58. The article of manufacture of claim 57 wherein the CD20 specific antibody is humanized 2H7 Variant A comprising a full length L chain of SEQ ID NO: 6 and a full length H chain of SEQ ID NO: 7, or a fragment thereof.

59. The article of manufacture of claim 58 wherein said humanized 2H7 Variant A is formulated at 20 mg/mL antibody in 10 mM histidine sulfate, 60 mg/ml sucrose, 0.2 mg/ml polysorate 20, and Sterile Water for Injection, at pH 5.8.

60. A pharmaceutical formulation comprising a complete effective amount of a CD20 specific antibody in a form suitable for one-time intravenous administration.

61. The pharmaceutical formulation of claim 60 wherein the CD20 specific antibody is humanized 2H7 Variant A comprising a full length L chain of SEQ ID NO: 6 and a full length H chain of SEQ ID NO: 7, or a fragment thereof.

62. The pharmaceutical formulation of claim 61 wherein the effective amount of said CD20 specific antagonist is between 400 mg and 1500 mg.

63. The pharmaceutical formulation of claim 60 wherein the CD20 specific antibody is humanized 2H7 Variant A comprising a full length L chain of SEQ ID NO: 6 and a full length H chain of SEQ ID NO: 7, or a fragment thereof.

64. The pharmaceutical formulation of claim 63 wherein said humanized 2H7 Variant A is formulated at 20 mg/mL antibody in 10 mM histidine sulfate, 60 mg/ml sucrose, 0.2 mg/ml polysorate 20, and Sterile Water for Injection, at pH 5.8.


SEQUENCE LISTING (15 sequences)

SEQ ID NO: 1 - protein, 107 aa, Artificial sequence
  Humanized antibody 2H7
DIQMTQSPSSLSASVGDRVTITCRASSSVSYMHWYQQKPGKAPKPLIYAPSNLASGVPSRFSGSGSGTDFTLTISSLQPEDFATYYCQQWSFNPPTFGQGTKVEIKR

SEQ ID NO: 2 - protein, 122 aa, Artificial sequence [sequence omitted]

SEQ ID NO: 3 - protein, 107 aa, Artificial sequence
  Humanized antibody 2H7
DIQMTQSPSSLSASVGDRVTITCRASSSVSYLHWYQQKPGKAPKPLIYAPSNLASGVPSRFSGSGSGTDFTLTISSLQPEDFATYYCQQWAFNPPTFGQGTKVEIKR

SEQ ID NO: 4 - protein, 122 aa, Artificial sequence
  Humanized antibody 2H7
EVQLVESGGGLVQPGGSLRLSCAASGYTFTSYNMHWVRQAPGKGLEWVGAIYPGNGATSYNQKFKGRFTISVDKSKNTLYLQMNSLRAEDTAVYYCARVVYYSASYWYFDVWGQGTLVTVSS

SEQ ID NO: 5 - protein, 122 aa, Artificial sequence
  Humanized antibody 2H7
EVQLVESGGGLVQPGGSLRLSCAASGYTFTSYNMHWVRQAPGKGLEWVGAIYPGNGATSYNQKFKGRFTISVDKSKNTLYLQMNSLRAEDTAVYYCARVVYYSYRYWYFDVWGQGTLVTVSS

SEQ ID NO: 6 - protein, 213 aa, Artificial sequence
  Humanized antibody 2H7
DIQMTQSPSSLSASVGDRVTITCRASSSVSYMHWYQQKPGKAPKPLIYAPSNLASGVPSRFSGSGSGTDFTLTISSLQPEDFATYYCQQWSFNPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - protein, 452 aa, Artificial sequence
  Humanized antibody 2H7
EVQLVESGGGLVQPGGSLRLSCAASGYTFTSYNMHWVRQAPGKGLEWVGAIYPGNGDTSYNQKFKGRFTISVDKSKNTLYLQMNSLRAEDTAVYYCARVVYYSNSYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 452 aa, Artificial sequence
  Humanized antibody 2H7
EVQLVESGGGLVQPGGSLRLSCAASGYTFTSYNMHWVRQAPGKGLEWVGAIYPGNGDTSYNQKFKGRFTISVDKSKNTLYLQMNSLRAEDTAVYYCARVVYYSNSYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNATYRVVSVLTVLHQDWLNGKEYKCKVSNKALPAPIAATISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 213 aa, Artificial sequence
  Humanized antibody 2H7
DIQMTQSPSSLSASVGDRVTITCRASSSVSYLHWYQQKPGKAPKPLIYAPSNLASGVPSRFSGSGSGTDFTLTISSLQPEDFATYYCQQWAFNPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - protein, 452 aa, Artificial sequence
  Humanized antibody 2H7
EVQLVESGGGLVQPGGSLRLSCAASGYTFTSYNMHWVRQAPGKGLEWVGAIYPGNGATSYNQKFKGRFTISVDKSKNTLYLQMNSLRAEDTAVYYCARVVYYSASYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNATYRVVSVLTVLHQDWLNGKEYKCKVSNKALPAPIAATISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 452 aa, Artificial sequence
  Humanized antibody 2H7
EVQLVESGGGLVQPGGSLRLSCAASGYTFTSYNMHWVRQAPGKGLEWVGAIYPGNGATSYNQKFKGRFTISVDKSKNTLYLQMNSLRAEDTAVYYCARVVYYSASYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNATYRVVSVLTVLHQDWLNGKEYKCAVSNKALPAPIEATISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 452 aa, Artificial sequence
  Humanized antibody 2H7
EVQLVESGGGLVQPGGSLRLSCAASGYTFTSYNMHWVRQAPGKGLEWVGAIYPGNGATSYNQKFKGRFTISVDKSKNTLYLQMNSLRAEDTAVYYCARVVYYSASYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNATYRVVSVLTVLHQDWLNGKEYKCKVSNAALPAPIAATISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 452 aa, Artificial sequence
  Humanized antibody 2H7
EVQLVESGGGLVQPGGSLRLSCAASGYTFTSYNMHWVRQAPGKGLEWVGAIYPGNGATSYNQKFKGRFTISVDKSKNTLYLQMNSLRAEDTAVYYCARVVYYSASYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNATYRVVSVLTVLHQDWLNGKEYKCKVSNAALPAPIAATISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHWHYTQKSLSLSPGK

SEQ ID NO: 14 - protein, 452 aa, Artificial sequence
  Humanized antibody 2H7
EVQLVESGGGLVQPGGSLRLSCAASGYTFTSYNMHWVRQAPGKGLEWVGAIYPGNGATSYNQKFKGRFTISVDKSKNTLYLQMNSLRAEDTAVYYCARVVYYSYRYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNATYRVVSVLTVLHQDWLNGKEYKCKVSNAALPAPIAATISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 452 aa, Artificial sequence
  Humanized antibody 2H7
EVQLVESGGGLVQPGGSLRLSCAASGYTFTSYNMHWVRQAPGKGLEWVGAIYPGNGDTSYNQKFKGRFTISVDKSKNTLYLQMNSLRAEDTAVYYCARVVYYSNSYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNATYRVVSVLTVLHQDWLNGKEYKCKVSNAALPAPIAATISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK